(19) United States
(12) Patent Application Publication
(10) Pub. No.: US 2008/0070264 A1
(43) Pub. Date: Mar. 20, 2008

(54) Intracellular Signaling-Induced Ret-Independent Gdnf Receptor-Effected Morphological Changes

(75) Inventors: Hannu Sariola, Helsinki (FI); Anna E. Popsueva, Helsinki (FI); Mart Saarma, Helsinki (FI)

Correspondence Address: WOODCOCK WASHBURN LLP, CIRA CENTRE, 12TH FLOOR, 2929 ARCH STREET, PHILADELPHIA, PA, 19104-2891, US

(73) Assignee: JPMORGAN CHASE BANK N.A., NEW YORK NEW YORK (NY)

(21) Appl. No.: 10/548,147

(22) PCT Filed: Mar. 5, 2004
(86) PCT No.: PCT/IB04/00985

§ 371 Date: May 23, 2006

(30) Foreign Application Priority Data
Mar. 5, 2003 (US) 60452219

(51) Int. Cl. G01N 33/574; C12Q 1/02

(52) U.S. Cl. 435/7.23; 435/29; 435/7.21

(57) ABSTRACT

The invention provides methods for identifying modulators of RETindependent, GDNF effected intracellular signaling. The invention further provides methods of identifying useful modulators of RET-independent GDNF receptoreffected MET activation, as well as modulators of morphological responses which are effected by nonRET GDNF receptor-effected intracellular signaling. Methods of distinguishing RET-independent GDNF ligand-mediated intracellular signaling by different cellular GDNF receptors from RET-dependent intracellular signaling are provided. The methods of the invention also provide an understanding of the complex interrelationships between RET, MET, GFRa in response to GDNF ligands.

### CROSS-REFERENCE TO RELATED APPLICATIONS

This claims benefit of U.S. Provisional Application No. 60/452,219 filed Mar. 5, 2003, the entirety of which is incorporated by reference herein.

### FIELD OF THE INVENTION

The present invention relates to cell signaling effected by GDNF and its receptors, and is more particularly related to methods for screening for modulators of RET-independent intracellular signaling effected by nonRET receptors, for example, GFRα receptors.

### BACKGROUND OF THE INVENTION

Glial cell line-derived neurotrophic factor (GDNF) (Lin et al., 1993), neurturin (NTN) (Kotzbauer et al., 1996), persephin (PSP) (Milbrandt et al., 1998) and a recently discovered artemin (ART) (Baloh et al., 1998) form a group of TGF-β family-related neurotrophic proteins. Studies in primary neuronal cultures, as well as in lesioned animal models, have provided evidence that GDNF is a survival factor for embryonic midbrain dopaminergic neurons (Beck et al., 1995; Lin et al., 1993; Tomac et al., 1995), spinal motor neurons (Henderson et al., 1994; Oppenheim et al., 1995; Yan et al., 1995), locus coeruleus noradrenergic neurons (Arenas et al., 1995), and subpopulations of peripheral sensory, sympathetic, and parasympathetic neurons (Buj-Bello et al., 1995; Trupp et al., 1995; reviewed by Airaksinen et al., 1999 and Saarma & Sariola, 1999). The pattern of neurotrophic activity of GDNF is therefore promising for its potential use in the treatment of Parkinson disease, Alzheimer disease, motoneuron diseases and several other neurodegenerative diseases. GDNF is also known to regulate ureteric branching and spermatogenesis, as well as survival and differentiation of several neuronal populations (see generally Airaksinen et al., 1999; Sariola and Saarma, 1999; Baloh et al., 2000; Meng et al., 2000).

The biological importance of the GDNF family is illustrated by the phenotype of GDNF null mice which display deficits in primary sensory, sympathetic and motor neurons. These mice also fail to develop kidneys and most of the enteric nervous system and they die at birth (Moore et al., 1996; Pichel et al., 1996; Sanchez et al., 1996). Despite its potential clinical importance, the intracellular mechanisms of GDNF's actions, and the GDNF receptor family are far from understood.

The receptor complex for GDNF consists of RET receptor tyrosine kinase (Durbec et al., 1996; Trupp et al., 1996), and glycosylphosphatidylinositol (GPI)-linked GDNF family receptor α1 (GFRα1) (Jing et al., 1996; Treanor et al., 1996).

In the embryonic kidney, GDNF is expressed by the metanephric mesenchyme and is repressed by epithelial conversion of the mesenchymal cells (Hellmich et al., 1996; Suvanto et al., 1996). GDNF-releasing beads stimulate ureteric branching in cultured kidneys and promote outgrowth of ectopic ureteric buds from the nephric duct (Sainio et al., 1997). Neutralizing antibodies to GDNF inhibit ureteric branching morphogenesis in vitro (Vega et al., 1996).

GFRα1, lacking an intracellular domain, was initially considered as only a ligand-binding receptor for GDNF, serving only in the presentation of the GFRα1/GDNF complex to RET (Jing et al., 1996; Treanor et al., 1996; Trupp et al., 1997). When complexed with two molecules of GDNF, a GFRα1 dimer induces dimerization of RET, recruitment of RET to lipid rafts, and transphosphorylation of the tyrosine kinase domains.

Lipid rafts are microdomains within cell membranes, consisting of sphingolipids and cholesterol, packed into moving platforms within the lipid bilayer (Harder et al., 1998). The raft microdomains serve as signalling compartments of the cell membrane, concentrating raft-specific signalling molecules (Simons and Toomre, 2000). RET is also activated in trans by GDNF via soluble or matrix bound GFRα1 (Paratcha et al., 2001). Moreover, GDNF signalling via RET differs inside and outside the lipid rafts (Saarma, 2001).

Although GPI-anchored membrane proteins have not been conclusively shown to exhibit independent intracellular signaling functions, evidence suggesting this possibility has been increasing (Simons and Ikonen, 1997; Friedrichson and Kurzchalia, 1998; Harder et al., 1998; Varma and Mayor, 1998; Viola et al., 1999). It has been shown, for example, that GPI-anchored proteins in the immune system can mediate intracellular signaling events, such as activation of the small G-proteins and Src-type tyrosine kinases, as well as elevation of intracellular free calcium concentration ([Ca²⁺]ᵢ) (Green et al., 1997; Brown and London, 1998; Viola et al., 1999).

RET and GPI-anchored GFRα1 are believed necessary receptors for GDNF (Cacalano et al., 1998; Enomoto et al., 1998), since mice lacking RET, GDNF or GFRα1 all share a similar phenotype and die soon after birth (see above). GDNF can also signal via GFRα1 in a RET-independent manner (Poteryaev et al., 1999; Trupp et al., 1999). In primary sensory neurons isolated from RET-deficient mice and in a RET-negative neuroblastoma cell line, GDNF activates Src-type kinases (Poteryaev et al., 1999; Trupp et al., 1999), however, it is not clear exactly how GFRα proteins evoke intracellular signals upon the action of GDNF family proteins in the absence of RET. It has also been shown, both in vitro and in vivo, that GDNF promotes survival of postnatal cochlear sensory neurons expressing GFRα1 mRNA but lacking RET mRNA, thus providing further evidence of RET-independent signaling triggered by activation of GRFα receptors (Ylikoski et al., 1998).

The triggering of GDNF-dependent intracellular signaling in RN33B cells has also been described (PCT/US96/18197, incorporated herein by reference). RN33B cells were described therein as expressing receptors for GDNF family ligands, none of which was c-RET. Two of the receptors were later determined to be GFRα1 and GFRα2 (reported as GDNFRα and GDNFRβ, respectively, U.S. patent application Ser. No. 08/861,990, incorporated herein by reference). The mechanism of the RET-independent signaling, however, was not known or described.

In another line of evidence, RET and GFRα1 expression patterns, while similar in some tissues or cell types, exhibit substantial differences in many tissues (Trupp et al., 1997; Enomoto et al., 1998, Golden et al., 1999, Kokaia et al., 1999). The different expression patterns of the GDNF receptors provides support for the existence of the distinct signaling of GFRα receptors alone, or, in conjunction with RET tyrosine kinase in trans (Yu et al., 1998).

Developmental differences are also observed. In kidney development, for example, ret is initially expressed along the nephric duct and the ureteric bud (Pachnis et al., 1993), and the receptor becomes restricted to the growing tips of the bud as branching progresses. gfrα1 is expressed by both ureteric bud and pretubular nephrogenic mesenchyme (Sainio et al., 1997). Targeted disruption of ret, gdnf or gfrα1 genes results in severe renal hypodysplasia or aplasia (Schuchardt et al., 1994; Pichel et al., 1996Sanchez et al., 1996; Cacalano et al., 1998) apparently confirming the critical role of GDNF/RET signalling in the ureteric branching.

Experimental models of ureteric branching are available. MDCK dog kidney epithelial cells have been extensively used for studying the molecular mechanisms of branching morphogenesis. While GDNF and HGF (hepatocyte growth factor) are both multifunctional signalling molecules with roles in embryogenosis, HGF binds to and activates MET receptor tyrosine kinase (Naldini et al., 1991). In vivo, HGF is required for early development of liver, limb muscles and placenta, and it is involved in liver regeneration (Birchmeier and Gherardi, 1998). In organ culture, HGF regulates ureteric bud branching and modulates epithelial differentiation of metanephric mesenchymal cells (Sainio et al., 1997; Karp et al., 1994; Wolf et al., 1995). In MDCK cells HGF induces scattering, chemotactic movements, and tubule formation (Stoker et al., 1987; Montesano et al., 1991). In the presence of soluble GFRα1, RET-transfected MDCK cells respond to GDNF like the wild-type MDCK cells respond to HGF (Tang et al., 1998).

In summary, the indirect evidence that GFRα1 has RET-independent functions in vivo include the following: (i) GDNF binds to GFRα1 in the absence of RET (Jing et al., 1996); and (ii) ret and gfrα1 expression patterns do not overlap in many tissues (e.g. Sainio et al., 1997; Golden et al., 1999). However, the mechanism(s) and the possible biological event(s), including morphological changes, regulated by RET-independent, GFRα-dependent signaling mediated by GDNF ligands, have previously remained unknown

There is a need in the art, therefore, for further elucidation of, RET-independent intracellular signaling and its mechanisms. In particular, methods for identifying compounds which modulate specific RET-independent cell signaling are needed.

### SUMMARY OF THE INVENTION

The present invention provides methods for screening for compounds that are modulators of nonRET GDNF receptor-mediated intracellular signaling, such as GFRα-dependent, RET-independent intracellular signaling. Compounds thus discovered are useful, for example, in preventing and treating conditions and diseases relating to altered RET-independent intracellular signaling.

In one aspect, the present invention relates to methods for identifying a compound which modulates RET-independent intracellular signaling effected by a nonRET GDNF receptor in a cell, comprising the steps of: incubating a cell expressing a nonRET GDNF receptor with a test compound; measuring an indicator of MET activation; and comparing the measured indicator to a measured indicator of MET activation from a control assay incubated without the test compound, thereby identifying whether the compound modulates RET-independent intracellular signaling.

The methods in certain preferred embodiments use a RET-independent GDNF-receptor effected morphological response as an indicator of MET activation. Preferred nonRET receptors include, but are not limited to, GFRα receptors. Also preferred for use with the present invention are certain cell types, for example, kidney cells, nervous system cells, fibroblasts and epithelial cells. The cells preferably exhibit a phenotype consistent with having no functional RET in some embodiments.

In another aspect, the present invention provides methods of identifying a modulating compound as above, wherein the indicator of MET activation is a RET-independent, GDNF-receptor effected, morphological response and wherein the method further comprises the step of comparing the measured morphological response to that of a control assay incubated under conditions which provide a RET-dependent GDNF receptor-effected morphological response, thereby further identifying a compound that modulates RET-independent intracellular signaling, RET-dependent intracellular signaling, or a combination of RET-independent and RET-dependent signaling.

In yet another aspect, the invention provides a method of identifying a compound that modulates RET-independent intracellular signaling effected by a nonRET GDNF receptor in a cell, comprising the steps of incubating a cell expressing a nonRET GDNF receptor with a test compound; measuring a RET-independent GDNF receptor-effected morphological response; and comparing the measured response to that of a control assay incubated without the test compound, thereby identifying whether the compound modulates RET-independent intracellular signaling.

In another of its aspects, the invention provides methods for studying complex interaction of receptors with a shared ligand. Provided are methods of differentiating one or more intracellular signals as effected by one or more receptors for a shared ligand comprising the steps of interacting the receptor(s) with a ligand at a first concentration for a first duration; measuring a change in response to the ligand in a first intracellular signal after the first duration; optionally, continuing to periodically measure changes in the first intracellular signal; further interacting the receptor(s) with the ligand at a second concentration for a second duration; wherein the second concentration is measurably higher than the first concentration and the second duration is at least as long as the first duration; measuring a change in response to the ligand in a second intracellular signal after the second duration; and, correlating the first intracellular signal with the first concentration and first duration and the second intracellular signal with the second concentration with the second duration, thereby differentiating the intracellular signals as effected by the receptor(s).

In another aspect of the invention, methods are provided for determining, in a cell, interrelationships between MET, RET and Src kinases mediated by multifunctional GDNF ligands. The methods comprise the steps of incubating a cell with a GDNF ligand at a concentration to be tested under conditions to be tested; measuring the activation of Src kinases and MET; correlating the activation of the Src kinases and MET with the concentration of GDNF ligand tested, and with the conditions to be tested, wherein the conditions to be tested include but are not limited to presence or absence of RET-dependent activation mechanisms, presence or absence of HGF ligand-mediated activation of MET, presence or absence of inhibitors of Src kinase; time course of the activation, cell genotype, and cell phenotype; thereby determining the interrelationships between MET, RET and Src kinases as mediated by GDNF ligands.

These and other features, advantages, and aspects of the present invention will be better understood by reference to the figures and detailed description that follow.

### BRIEF DESCRIPTION OF THE DRAWINGS

FIG. 1. Wild-type MDCK cells do not express endogenous ret.

FIG. 1A: RT-PCR with canine specific ret primers; cyclophilin was used as a control for equal loading.

FIG. 1B: Northern blotting.ret transcript is detected in mRNA from normal dog testes but not from MDCK cells.

FIG. 2. GDNF induces branching of GFRα1-expressing MDCK cells in 3-D collagen gels.

FIG. 2A: MDCK cells expressing RET/GFRα1 or expressing GFRα1 but not RET, were grown in collagen gel with GDNF (100 ng/ml). Wild-type MDCK cells were grown with HGF (50 ng/ml). BSA (10 mg/ml) was used as a negative control.

FIG. 2B: GDNF (100 ng/ml) induces branching of GFRα1 and RET/GFRα1 cells but not wild-type MDCK cells, which only branch on exposure to HGF (50 ng/ml) stimulation. PSPN (100 ng/ml) does not induce branching of any MDCK cell lines. From the total amount of cysts in the field, the percentage of cysts with long branches was calculated. Only branches with lengths of more than two cyst diameters were counted.

FIG. 2C: Dose-dependence of GDNF-induced branching of GFRα1- and RET/GFRα1-expressing MDCK cells. GDNF concentrations are per ml (x-axis). Results are reported as fold of branching cysts count over the non-induced control. Means±standard errors of the mean of five to eight counted fields are shown. The results are representative of five (A and B) and three (C) independent experiments, p<0.001.

FIG. 3. GFRα1-expressing, RET-deficient MDCK cells do not show a chemotactic response to GDNF.

FIG. 3A: In Boyden chamber chemotaxis assays, mock-transfected, GFRα1-expressing and RET/GFRα1-expressing cells were exposed to GDNF, and wild-type MDCK to HGF. The number of cells was counted as described in Materials and Methods. +/+=GDNF at 100 ng/ml or HGF at 50 ng/ml was added to both chambers to assay chemokinesis. Each result represents the mean±SEM (n=3). For each case, p<0.001.

FIG. 3B: Chemoattraction assay on collagen matrix. Only RET/GFRα1-expressing cells migrate towards GDNF-soaked agarose beads. BSA-soaked agarose beads were used as negative controls. Agarose beads are denoted by white circles. Note that mock transfected, GFRα1-expressing and RET/GFRα1-expressing cells form clusters of adherent cells (denoted by arrowhead) with BSA-soaked beads after 3 days, whereas RET/GFRα1 cells migrating towards GDNF bead are dissociated.

FIG. 4. GDNF induces phosphorylation of MET.

FIGS. 4A, 4B: Dose-dependent phosphorylation of MET by GDNF in GFRα1 -expressing and Ret/GFRα1-expressing MDCK cells. MET was activated in 15 minutes after GDNF application. The lower panels show the re-probing of the same filter with anti-MET antibodies. The numbers below the lanes indicate the fold of induction of MET tyrosine kinase.

FIG. 4C: Induction of MET in mock transfected MDCK cells. Concentrations of GDNF and HGF are given in ng/ml. 30 μg of total protein from each sample (i.e. equal loading) were incubated with 10 μl of immobilized phospho-tyrosine monoclonal antibodies. Immunocomplexes were washed and analysed as described under “Materials and Methods”.

FIG. 4D: Dose-dependent activation of RET by GDNF in RET/GFRα1-expressing MDCK cells. RET was not activated until 2 hours after GDNF application. The lower panel shows the re-probing of the same filter with anti-RET antibodies. The numbers below the lanes indicate the fold of induction of RET tyrosine kinase. IP, immunoprecipitation; WB, Western blotting; P-tyr, phospho-tyrosine. The results are representative of three independent experiments.

FIG. 5. The co-expression of gfrα1 and met mRNA in mouse E15 metanephric kidney and adult epididymis.

FIG. 5A: The expression of gfrα1 and met overlap in the kidney cortex, pretubular mesenchyme (indicated by arrows) and the ureteric bud epithelium (indicated by asterisks) of the metanephric kidney. Note that ret is expressed only in the tips of the ureteric buds. Inset figures show high magnification view of a ureteric tip.

FIG. 5B: Sections from the initial segment of epididymis. Scale bar: 100 μm.

FIG. 6. GFRα1 does not complex with MET.

Binding of ¹²⁵I-GDNF to COS7 cells transfected with GFRα1, and ¹²⁵-HGF to wild-type COS7, followed by cross-linking with EDC together with sulfo-NHS. Inmunoprecipitates with anti-MET antibodies (IP-Met) were analysed by SDS-PAGE under reducing conditions. ¹²⁵I-HGF α subunit and proHGF are marked by arrows. ¹²⁵I-HGF/MET complexes are denoted by arrow heads. The results are representative of five independent experiments.

FIG. 7. GDNF-induced activation of MET and branching tubulogenesis in GFRα1-expressing and RET/GFRα1-expressing cells requires Src kinases.

FIG. 7A: Dose-dependent Src-kinase activation by GDNF in GFRα1-expressing and RET/GFRα1-expressing MDCK cells. The activation of Src-type kinases was observed after 15 minutes. The concentrations of GDNF are indicated. The numbers below the lane reflect the fold of increase in phosphorylation of Tyr418 of Src. The lower panels show the re-probing of the same filter with anti-Src antibodies. The results are representative of three independent experiments.

FIG. 7B: SHEP cells were grown with GDNF (10 ng/ml (lanes 2-4) or 5 pg/ml (lanes 5-7)) or HGF (10 ng/ml (lanes 8-9)) in the presence of PP2 (1 μM (lanes 3 and 6) and 10 μM (lanes 4 and 7)). To exclude a possible cytotoxic effect of the solvent, DMSO was added to the controls. The lower panel shows the re-probing of the same filter with anti-MET antibodies. The numbers below the lane indicate the fold of induction of MET tyrosine kinase. The results are representative of three independent experiments.

FIG. 8. Exogenous GDNF partially restores ureteric branching of ret-deficient kidneys.

FIG. 8A-FIG. 8D: Urogenital blocks including the Wolffian duct (wd), mesonephros (meso) and metanephros (meta) were dissected from E11 ret −/− (panels A, B), ret +/− (panel C) and ret +/+ (panel D) mouse embryos. The two urogenital blocks from the embryos were separately cultured for 4 days. One side was cultured without GDNF (panels A, C, D) and the other side with 50 ng/ml of GDNF (panel B). Urogenital block explants were fixed and immunostained with pan-cytokeratin antibodies. Scale bar: 200 μm.

FIG. 8E: The number of ureteric branches of ret −/−, ret +/− and ret +/+ kidneys with or without GDNF supplementation. Numbers in parenthesis indicate the kidney explants counted. Since exogenous GDNF did not reduce the number of ret −/− urogenital explants without any ureteric bud (see Table I), the number of ureteric branches in ret −/− kidneys is from the explants that showed ureteric buds and branches. Each result represents the mean±SEM (n=5). Exogenous GDNF significantly increased ureteric branching in ret −/− explants compared to the control media (p<0.05).

### DETAILED DESCRIPTION

A. Overview

The invention, in its several aspects, provides methods of identifying modulators of RET-independent GDNF receptor-effected intracellular signaling. The invention provides insights into the complex interrelationships between receptors and ligands in both the traditional upstream and downstream activation pathways, as well as cross-talk and horizontal activation of intracellular signaling components. These insights have contributed to the development of the useful methods disclosed herein for identifying compounds which modulate intracellular signaling, in particular, RET-independent GDNF receptor-effected intracellular signaling. The methods provided herein will be further described in detail below. It will be appreciated that the description of these complex receptor interrelationship is necessarily limited by language, and thus, the invention as described is capable of variations and modifications such as are in keeping with the spirit of the appended claims.

B. Definitions

Various terms relating to the methods, biological molecules and cells of the present invention are used throughout the specifications and claims.

“A”, “an”, and “the” as used herein refer to the singular and plural.

The terms “include” or “includes” or “included” as used herein refers to the nonlimiting sense of the term, unless otherwise indicated or unless construing the term as such would render meaningless a phrase wherein the term is used.

The term “effect” as used herein as a noun means an alteration or change. An effect can be positive, such as causing an increase in some material, or negative, e.g., antagonistic or inhibiting. However, where the term “effected” is used as in connection with a receptor, as in, for example, “receptor-effected intracellular signaling” the term means, for example capable of bringing about the referenced signal, or capable of causing the referenced signal to come into being under at least certain conditions.

The term “modulator” as used herein refers to a compound or a composition that is capable of altering the intracellular signaling pathways effected by a receptor, and includes, but is not limited to, agonists and antagonists. “Modulators” also includes analogs, mimetics and the like of a natural ligand. Modulators can work in concert or in opposition to other compounds or compositions which influence the intracellular signal, either before, during, or after an interaction of the ligand with the receptor, or the binding thereof.

The term “agonist” as used herein refers to a compound or composition that can stimulate or positively influence the intracellular signaling pathways of a receptor, or can supplement, augment or act synergistically with the activity of any other compound or composition thereon.

The term “antagonist” as used herein refers to a compound or composition that can inhibit, suppress, block or negatively influence the intracellular signaling pathways of a receptor.

The term “sufficient amount” as used herein refers to a quantity of an agent that will result in the referred to effect.

The term “bind” as used herein refers to the interaction between ligands and their receptors, the binding being of a sufficient strength and for a sufficient time to allow the recognition of said binding by the receptor, under the conditions of the assays disclosed herein.

The term “about” in reference to a numerical value means ±10% of the numerical value, more preferably±5%, most preferably±2%.

The term “administration” includes but is not limited to, oral, subbuccal, transdermal, parenteral, nasal, sublingual, subcutaneous and topical. A common requirement for these routes of administration is efficient and easy delivery.

As used herein, the term “effective amount,” refers to the amount required to achieve an intended purpose for both prophylaxis or treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3 In: Goodman & Gilman's The Pharmacological Basis of Therapeutics, 9th Ed., Hardman et al., eds., McGraw-Hill, New York, N.Y., 1996).

The term “nervous system cell” as used herein refers to any cell or cells present in or derived from the nervous system, including, but not limited to neuronal cells, such as neurons, and non-neuronal cells, such as glial cells.

The term “kidney cell” as used herein refers to any cell or cells present in or derived from the kidney of any species, including, but not limited to kidney-derived cells, such as, but not limited to MDCK cells, such as buffalo green monkey kidney cells or opossum kidney cells.

The term “kidney cell” also includes any cell which developmentally related to the kidney organ, or any cell which differentiates into a kidney cell, or which has differentiated from a kidney cell.

A cell has been “transfected” by exogenous or heterologous DNA when such DNA has been introduced inside the cell, and includes stably transfected cells, in which the exogenous DNA has become integrated into a chromosome so that it is inherited by daughter cells through chromosome replication. This stability is demonstrated by the ability to establish cell lines or clones comprised of a population of daughter cells containing the exogenous DNA. The transfected DNA may or may not be integrated (covalently linked) into the genome of the cell. In prokaryotes, yeast, and mammalian cells for example, the exogenous may be maintained on an episomal element such as a plasmid. A “clone” is a population of cells derived from a single cell or common ancestor by mitosis.

A “cell line” is a clone of a primary cell that is capable of stable growth in vitro for many generations. Methods of transfection are specific to the kinds of cells transfected and are well known in the art.

“Mock-transfected” cells are cells which are subjected to the procedure of transfection but into which no new is transferred, thus they are useful as controls to ensure the process has not altered the cells not so transfected.

The term “kidney disease,” as used herein, means any disturbance in structure or function of any kidney cell, from whatever cause, and shall include all abnormalities, whether originating genetically or environmentally, present congenitally or later acquired, and from any cause, whether infectious, traumatic, toxic, degenerative, inflammatory or neoplastic. This shall include but not be limited to any degenerative or retrogressive process within one or more cells of the kidney regardless of the extent of such degeneration or retrogression.

The term “cellular response” as used herein refers to, without limitation, any change in cell survival, cellular plasticity, cellular extension, cell migration, or any enhancement or inhibition of cellular activities.

The term “lipid rafts,” as used herein, refers to a structure of sphingolipids and cholesterol packed into moving platforms within the liquid bilayer of cell membranes, and includes the detergent insoluble, glycolipid-enriched fraction that remains after extraction with Triton X-100 or similar detergents.

The terms “GPI-anchored” or “GPI-linked” as used herein in reference to a receptor refer to a receptor that is associated with GPI.

As used herein in reference to a receptor, the term “-independent intracellular signaling” refers to a receptor that evokes intracellular signaling in the absence of the receptor so referenced, or without a requirement for the referenced receptor.

The term “nonRET,” as used herein, refers to a protein other than RET, in particular, reference to a “nonRET” GDNF receptor includes any receptor which recognizes, binds, responds, or generates a cellular signal in connection with exposure to GDNF ligands.

As used herein, “GDNF ligands” include GDNF, as well as any compound or composition which possesses GDNF activity. The term also includes analogs, derivatives, salts, mimetics or other compounds related to GDNF or which are recognized by GDNF receptors, or which effect intracellular signaling by a GDNF receptor.

A phenotype is “consistent” with a particular characteristic, for example, when the phenotype has several routes by which it can be achieved. A phenotype is “consistent” with having no functional RET when the cell, regardless of its genotype or its complement of receptors does not exhibit functional RET protein. Cells lacking a functional ret gene, expressing a defective RET protein, or expressing a potentially functional RET protein that is rendered nonfunctional by some other cellular condition, including growth conditions, are examples cells with phenotypes consistent with having no functional RET, regardless of the cell's genotype.

The term “morphological response” as used herein includes any observable change in the morphology of a cell, whether macroscopic or microscopic. The term “morphological response” also includes chemotaxis (i.e. guided migration of cells) and chemokinesis (i.e. enhanced but random movement of cells).

Various techniques of molecular biology are used herein. The techniques are known to those of skill in art. Standard treatises on techniques of cloning, PCR and related techniques, transformation, are known to those of skill in the art. For a general overview of PCR see PCR Protocols: A Guide to Methods and Applications. Innis, M, Gelfand, D., Sninsky, J. and White, T., eds, Academic Press, San Diego (1990). Those of skill in the art will generally appreciate standard protocols such as those set forth in current editions of “Current Protocols in Molecular Biology”, eds. Frederick M. Ausubel et al., John Wiley & Sons, 1999 or “Molecular Cloning: A Laboratory Manual” Sambrook, J., Fritsch, E. F., and Maniatis, T., Cold Spring Harbor Laboratory Press, NY, Vol. 1, 2, 3 (1989) and the like. In addition, the manufacturers of the various reagents and kits cited in the “Materials and Methods” provide particularized instructions as to the methods for use of their products as used herein.

Unless otherwise indicated, abbreviations used herein areas follows: GDNF, glial cell line-derived neurotrophic factor; HGF, hepatocyte growth factor; GFRα1, GDNF family receptor α1; GPI glycosylphosphatidylinositol; GFP, green fluorescent protein; FCS, fetal calf serum; PBS, phosphate-buffered saline; BSA, bovine serum albumin; RT-PCR, reverse transcription-polymerase chain reaction; PSPN, persephin; E, embryonic day.

C. Detailed Description:

Novel methods for identifying useful modulators of nonRET GDNF receptor-mediated intracellular signaling, such as GFRα-dependent, RET-independent intracellular signaling, are provided herein. The modulators thus identified are useful in preventing and treating conditions and diseases relating to altered RET-independent intracellular signaling, and may have utility in treating cancer and other disease states, thus the methods of the present invention also have utility in identifying therapeutic compounds relating to RET-independent intracellular signaling.

In a first aspect, the present invention relates to methods for identifying a compound which modulates RET-independent intracellular signaling effected by a nonRET GDNF receptor in a cell, comprising the steps of: incubating a cell expressing a nonRET GDNF receptor with a test compound; measuring an indicator of MET activation; and comparing the measured indicator to a measured indicator of MET activation from a control incubated without the test compound, thereby identifying whether the compound modulates RET-independent intracellular signaling.

The nonRET GDNF of the methods of the invention may be any receptor, which is not RET itself, and which can effect intracellular signaling upon exposure to a GDNF ligand. NonRET GDNF receptors may be soluble or membrane-associated. Presently preferred are nonRET GDNF receptors which are membrane-associated. More preferred are nonRET GDNF receptors that are GPI-anchored. Also preferred are nonRET GDNF receptors that are capable of associating with lipid rafts. In a presently preferred embodiment, the nonRET GDNF receptor is a GFRα family protein. In highly preferred embodiments, the nonRET GDNF receptor is a GFRα1.

Several indicators of MET activation are well-known to those of skill in the art. It will be appreciated that any such indicator is appropriate for use with the methods disclosed herein. Preferred embodiments employ indicators which are for example, accurate, inexpensive or easy to use. In a presently preferred embodiment, the methods use a RET-independent GDNF receptor-effected morphological response as the indicator of MET activation. Presently preferred morphological responses include, but are not limited to tubulogenesis and branching tubulogenesis. Such morphological responses are relatively easy to measure, as they can frequently be observed in light microscopes as are available in the art. Counting methods for counting branching or tubule formation are straight-forward and do not required expensive equipment. Other cellular changes reflective of morphological responses for use herein include chemotactic and chemokinetic responses. These changes, too, are simple to measure, and are also reflective in detail of intracellular signals which have effect on the cell as a whole. Those of skill in the art will appreciate that chemotaxis and chemokinesis can be measured by a variety of techniques, for example, in a Boyden chamber.

Also preferred for use with the present invention are certain cell types, for example, kidney cells, nervous system cells, fibroblasts and epithelial cells. Testicular cells are also used for certain purposes as described in the examples. The cells may have oncogenic or other mutations that alter the expression of one or more cellular receptors involved in intracellular signaling. Oncogenic mutations relating to cellular receptors are well-known in the art. In more preferred embodiments, the cell is a SHEP neuroblastoma cell, a COS7 cell, a NIH 3T3 cell, a MDCK dog kidney cell.

In a presently preferred embodiment of the methods taught herein, the incubated cell has a phenotype consistent with having no functional RET. It is to be appreciated that a variety of genotypes and conditions can result in such phenotypes. In some cases the cell is ret −/−, i.e. does not contain a gene encoding the RET protein. In other cases, the cell contains a defective gene, or alternatively a gene encoding a nonfunctional or defective RET. In preferred embodiments this RET is biologically inactive with respect to GDNF ligands in particular. Cells expressing nonfunctional RET are suitable with the methods of the present invention. In some embodiments, the cell expresses a RET which is partially or fully biological functional with respect to GDNF ligands. In such embodiments, the RET dependent and RET independent signaling can be distinguished on the basis of the kinetics of the responses at various concentrations of GDNF ligand, as in demonstrated in the examples. Cell phenotypes consistent with having no functional RET, as well as those consistent with having a functional RET may be naturally occurring or may result from genetic manipulations, for example, wherein the cells are cloned to express a nonRET GDNF receptor, or to eliminate a functional RET. Genetically manipulated cells for use with the present invention are either stably or transiently able to express the desired receptor(s). In addition, the genetically manipulated cells may express naturally-occurring or modified forms of the receptor(s), for example, fusion proteins between the receptor and other biologically useful proteins, such GFP, are known in the art. Such constructs have utility for use herein, for example, they can maintain functionality as receptors and also be readily visualized to confirm proper membrane localization. Those of skill in the art will appreciate that the cloned ret and gfrα1 genes and constructs disclosed herein are useful for such purposes as constructing cell lines for use with the methods of the invention.

The methods of the invention, in some embodiments, are practiced in the absence of a known GDNF ligand. In such methods, the test compound is typically being explored for activity as GDNF ligand or agonist. In other embodiments, the methods of the invention are practiced in the presence of a known GDNF ligand. In such methods the test compound may be tested for activity as a modulator of any type as defined herein. In particular, to test for antagonistic activity of a test compound, the methods must be practiced in the presence of a GDNF ligand which is known to trigger the intracellular signaling pathway which the receptor effects. Control assays are always included and the results with the test compounds are compared to these controls. In preferred embodiments, the RET-dependent cellular mechanisms requires a longer time and a higher concentration of GDNF ligand to effect intracellular signaling than the RET-independent mechanisms, and the GDNF ligand is added at a concentration below that required for the RET-dependent mechanisms, but at a concentration sufficient to effect RET-independent intracellular signaling. Presently preferred are methods wherein the GDNF ligand is present at a concentration of less than about 100 pg/ml, 10 pg/ml, 1 pg/ml, 100 fg/ml, or 50 fg/ml respectively.

Other means by which RET-dependent and RET-independent signals of the invention are disclosed herein. Contemplated for use with the present invention are methods wherein the cell produces biologically functional RET, but an inhibitor of the RET-dependent intracellular signaling is used to block such signals to an extent which allows measurement of the RET-independent signals. Methods are also disclosed wherein the incubation time is less than that required by RET-dependent GDNF-effected intracellular signals. Such signals have been shown to require on the order of two hours. Thus, in presently preferred embodiments of the invention, incubation time for the methods is less than about 2 h, 1 h, 30 min, 15 min, or 5 min respectively.

In another aspect, the present invention provides methods of identifying a modulating compound as above, wherein the indicator of MET activation is a RET-independent GDNF-receptor effected morphological response and wherein the method further comprising the step of comparing the measured morphological response to that of a control assay incubated under conditions which provide a RET-dependent GDNF receptor-effected morphological response, thereby further identifying a compound that modulates RET-independent intracellular signaling, RET-dependent intracellular signaling, or a combination of RET-independent and RET-dependent signaling.

In yet another aspect, the invention provides a method of identifying a compound that modulates RET-independent intracellular signaling effected by a nonRET GDNF receptor in a cell, comprising the steps of incubating a cell expressing a nonRET GDNF receptor with a test compound; measuring a RET-independent GDNF receptor-effected morphological response; and comparing the measured response to that of a control assay incubated without the test compound, thereby identifying whether the compound modulates RET-independent intracellular signaling.

In another of its aspects, the invention provides methods for studying complex interaction of receptors with a shared ligand. Provided are methods of differentiating one or more intracellular signals as effected by one or more receptors for a shared ligand comprising the steps of interacting the receptor(s) with a ligand at a first concentration for a first duration; measuring a change in response to the ligand in a first intracellular signal after the first duration; optionally, continuing to periodically measure changes in the first intracellular signal; further interacting the receptor(s) with the ligand at a second concentration for a second duration; wherein the second concentration is measurably higher than the first concentration and the second duration is at least as long as the first duration; measuring a change in response to the ligand in a second intracellular signal after the second duration; and, correlating the first intracellular signal with the first concentration and first duration and the second intracellular signal with the second concentration with the second duration, thereby differentiating the intracellular signals as effected by the receptor(s).

In another aspect of the invention, methods are provided for determining, in a cell, interrelationships between MET, RET and Src kinases mediated by multifunctional GDNF ligands. The methods comprise the steps of incubating a cell with a GDNF ligand at a concentration to be tested under conditions to be tested; measuring the activation of Src kinases and MET; correlating the activation of the Src kinases and MET with the concentration of GDNF ligand tested, and with the conditions to be tested, wherein the conditions to be tested include but are not limited to presence or absence of RET-dependent activation mechanisms, presence or absence of HGF ligand-mediated activation of MET, presence or absence of inhibitors of Src kinase; time course of the activation, cell genotype, and cell phenotype; thereby determining the interrelationships between MET, RET and Src kinases as mediated by GDNF ligands.

### EXAMPLES

### Materials and Methods

Kidney Cell Culture and Transfections

Early passage MDCK cells were cultured in MEM with 10% fetal calf serum (FCS). Human SHEP neuroblastoma cells and Neuro-2A cells were cultured in RPMI 1640 with 10% FCS. NIH 3T3 cells and COS7 cells were cultured in DMEM with 10% FCS, transiently transfected with pcDN3-GFRα1/GFP using FUGENE 6™ reagent (Roche). For creation of stable lines, MDCK cells were transfected in equal portions with pcDN3-RET, pcDN3-GFRα1, pcDN3-GFRα1/GFP using FUGENE 6™ reagent and selected with 400 μg/ml G418 (GibcoBRL, Life Technologies). After 2 weeks of selection, multiple clones were collected and the expression of ret and/or gfrα1 was verified by RT-PCR and Western blotting. RET/GFRα1 (N=7 and N=17), GFRα1 (N=14) and GFRα1-GFP (N=2 and N=3) clones which showed high level of exogenous protein expression according to the Western blot were used for further analyses.

GFP-GFRα1 Fusion Expression Plasmid Construction

Fusion proteins of GFP (green fluorescent protein) and GFRα1were created by cloning the coding sequences in a plasmid. The coding sequence (full-length except for the sequence corresponding to the first methionine) of GFP was amplified by PCR with primers: 5′-aattgctagcgtgagcaagggcgaggagc-3′ (SEQ ID NO: 1);5′-aattgctagcttacttgtacagctcgtcc-3′ (SEQ ID NO:2). The primers contained Nhe I restriction sites flanking the GFP sequence. The full-length GFRα1 coding sequence was cloned into pcDNA3 expression vector and subjected to “inverse PCR”. The “sense” GFRα1 primer with Nhe I (5′-aattgctagcgaccgtctggactgtgtgaaag-3′) (SEQ ID NO:3) was designed to anneal to the beginning of mature GFRα1 sequence, whereas the “antisense” primer with Nhe I (5′-tatagctagctccaccactcacctcggcgg-3′) (SEQ ID NO:4) annealed to the end of signal leader peptide of the GFRα1 precursor. The resulting PCR products were digested with Nhe I and ligated. The expression construct therefore is the N-terminal fusion of mature GFRα1 to GFP preceded by in-frame signal peptide of GFRα1 with starting methionine, which targets the fusion to the extracellular protein pathway. The membrane localization of the fusion was confirmed by confocal microscopy in transiently transfected SHEP and Neuro-2a cells. The ability of the construct to encode a functional receptor for GDNF was verified by MAPK activation assay in transiently transfected RET-positive Neuro-2a cells (data not shown).

Cloning of ret cDNA from Dog Testes

Total RNA was isolated from autopsy samples of adult dog testes with TRIZOL reagent (GibcoBRL, Life Technologies). Reverse transcription reaction was performed using SUPERSCRIPT II RT (GibcoBRL, Life Technologies). PCR (40 cycles) was performed with primers for human c-ret 5′-AGACGTGGTACCTGCATCAGG-3′ (SEQ ID NO:5), 5′-CGTTGAAGTGGAGCAAGAGG-3′ (SEQ ID NO:6). The PCR product was cloned into pGEM-T vector (Promega) and sequenced (GeneBank AF364316, (SEQ ID NO:7) incorporated by reference herein in its entirety).

Primers from nucleotides 29-49 (SEQ ID NO:8) and 225-244 (SEQ ID NO:9) of GeneBank sequence AF364316 (SEQ ID NO:7) were used in RT-PCR analysis for canine ret expression. For Northern blotting, 30 μg of total RNA per lane was separated in 1.2% formaldehyde-agarose gel and transferred by capillary blotting onto HYBOND-N membrane (Amersham Pharmacia Biotech) according to the manufacturer's instructions. Blots were hybridized with [³²P]dCTP-labeled canine ret probe (GeneBank AF364316, SEQ ID NO:7), then washed 2 times in 1×SSC/0.1% SDS buffer and 3 times in 0.5×SSC/0.1% SDS buffer +42° C. (Sambrook et al., 1989). Detection of the hybridized Northern blots was by Phosphoroimager, Fuji.

Western Blotting and Immunoprecipitation

To analyze Src activation, subconfluent GFRα1- and RET/GFRα1-expressing MDCK cell cultures were starved for 24 h before induction in serum-free MEM. After 10 min incubation at 37° C. with 50 ng/ml GDNF (Cephalon Inc. or R&D Systems, Inc.) or 50 ng/ml HGF (Sigma) cells were lysed in lysis buffer supplemented with 1 mM Na-orthovanadate and analyzed by Western blotting as described (Lindahl et al., 2001). The Western blots were probed with the indicated antibodies and developed with ECL reagents (Amersham Pharmacia Biotech). Antibodies used included anti-Y⁴¹⁸ Src and anti-Src (BioSource International).

To detect MET activation, GFRα1- and RET/GFRα1-expressing MDCK or SHEP cells were starved overnight in MEM or RPMI 1640 with 1% FCS, respectively, and in serum-free medium for 2 h prior the induction. After 15 min incubation at 37° C. with GDNF (at indicated concentrations) or with 50 ng/ml HGF, cells were lysed as described. Cleared cell lysates were incubated with anti-MET antibodies (Santa Cruz Biotechnologies, Inc.) overnight at 4° C. Immunoprecipitates were collected with protein A-SEPHAROSE (Amersham Pharmacia Biotech), washed, separated by SDS-PAGE and transferred to HYBOND-ECL membranes. Membranes were immunoblotted with anti-phospho-tyrosine antibodies (Upstate Group Inc.) or anti-MET antibodies.

The above procedure was also followed to detect RET phosphorylation in RET/GFRα1-expressing MDCK, however, for these experiments, anti-RET antibodies (Santa Cruz Biotechnologies, Inc.) were used for the immunoprecipitation and immunoblotting.

Alternatively, 30 μg of protein was incubated overnight at 4° C. with 10 μl Immobilized Phospho-Tyrosine Monoclonal Antibodies (Cell Signalling, NEB). Immunocomplexes were washed and analysed as described.

For all experiments, densitometry and quantifications were performed using TINA 2.0 program (Raytest, Straubenhardt, Germany).

For the inhibition of MET, and Src activation, SHEP or GFRα1- and RET/GFRα1-expressing MDCK cells were starved as described above. 1 or 10 μm of the inhibitor PP2 (Calbiochem) was added 30 min before induction by GDNF or HGF. DMSO (dimethyl sulfoxide) was added to the positive controls together with GDNF or HGF.

¹²⁵I-labelled GDNF and HGF Binding, Chemical Crosslinking

GDNF and HGF were enzymatically iodinated with [¹²⁵I]NaI (Amersham Pharmacia Biotech) with lactoperoxidase to a specific activity of 100.000 cpm/ng as described (31). COS7 cells were transfected with pc3-GFRα1/GFP two days prior the assay. 2 nM of ¹²⁵I-GDNF or 1 nM of ¹²⁵I-HGF were allowed to bind to cell monolayers for 1-2 h on ice in binding buffer (DMEM/15 mM HEPES, pH 7.5; 0.2% BSA), washed, and chemically cross-linked for 30 min at room temperature using BS³ ([Bis(sulfosuccinimidyl)suberate]), DSS ((Disulfosuccinimidyl)suberate), DSP (3,3-Dithio-bis-(sulfosuccinimidyl)propionate) or EDC ((1-Ethyl-3-[3-dimethylaminopropyl]carbodiimide Hydrochloride)) with sulfo-NHS (N-Hydroxysulfosuccinimide) (Pierce). The non-specific binding of GDNF was estimated by the amount of ¹²⁵I-GDNF binding to cells in the presence of 300 n M unlabeled GDNF. Cells were washed, lysed and immunoprecipitated with anti-MET antibodies as described. Gels were dried and analyzed by phosphorimaging in a BAS Reader 1800 (Fuji).

Cell Migration and Chemotaxis Assays

5×10⁴ MDCK cells (GFRα1-, RET/GFRα1-expressing and mock transfected) were suspended in 300 μ1 of MEM with 10% FCS and seeded into 24-well cell culture inserts with the filters (Boyden chambers), pore size 8 μm (Falcon). The assay was done as described (Tang et al., 1998). Briefly, GDNF or HGF was added to the upper, or both the upper and lower chambers at the marked concentrations. After 48 h incubation, non-migrated cells on the upper surface of the filters were scrapped. Membranes with the cells on the lower surface were washed with PBS, fixed by 3% glutaraldehyde in PBS, stained with May-Grünwald Giemsa (MGG) solution, dehydrated and mounted on the slides. Cells in eight fields of each membrane were counted under the light microscope (Magnification=100×). The average and standard error of the mean were calculated. Statistical significance of the differences was estimated by t-test.

3.5 cm dishes were coated with collagen I solution and 20,000 cells were seeded on the coated dish. GDNF-soaked agarose beads, prepared as described (Sainio et al., 1997), were put on the gel before it solidified. Cells around the beads were photographed daily.

Branching Tubule Formation Assay

Trypsinized cells were mixed 1:3 with collagen type I solution and plated. MEM with 10% FCS was overlaid on the gels with or without GDNF (Cephalon Inc. or R&D Systems, Inc.) or 50 ng/ml HGF. Cells in collagen were cultured for 3 days; GDNF-containing medium was changed daily. For quantification, cells were cultured for 3 days, fixed by 3% glutaraldehyde in PBS and counted under a light microscope.

To avoid the effect of possible contamination of GDNF preparation, recombinant GDNF from two different sources were tested. One tested GDNF was expressed in baculovirus-infected insect cells (Cephalon Inc.), the other was expressed in mouse myeloma cell line NSO (R&D Systems, Inc.).

In Situ Hybridization

Different developmental stages of embryonic kidneys from CBA×NMRI mice were dissected, fixed in 4% paraformaldehyde for 2-24 h and processed to paraffin. In situ hybridization was performed as described (Copp and Cockroft, 1991). 550 nt anti-sense and sense cRNA probes from mouse met (GenBank accession number Y00671) were provided by Dr. T. Timmusk and M. Lindahl (Institute of Biotechnology, University of Helsinki) and a 777 nt cRNA probe of mouse gfrα1 (GenBank accession number AB000800; Meng et al., 2000) was synthesized using appropriate RNA polymerases and ³⁵S-labeled UTP.

The examples and embodiments described herein are for illustrative purposes only and that various modifications or changes in light thereof will be suggested to persons skilled in the art and such are to be included within the spirit and purview of this application and scope of the appended claims. All publications, patents, and patent applications cited herein are hereby incorporated by reference in their entirety for all purposes.

### Example 1

GDNF induces branching of GFRα1-transfected/RET-deficient MDCK cells.

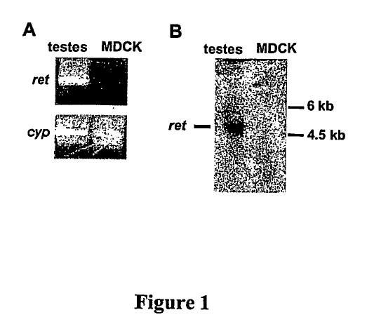

MDCK cells were transfected with expression vectors encoding the human ret together with rat gfrα1, or the gfrα1 only, with or without fused green fluorescent protein (GFP). Multiple clones expressing GFRα1 with and without fused GFP, and clones expressing both RET and GFRα1 were identified by RT-PCR and Western blotting. Clonal cell lines expressing GFRα1 with (n=3) or without GFP (n=14) showed similar biological responses to GDNF (see below and data not shown). The expression of ret in wild-type MDCK cells was not detectable by either Northern blot analysis or RT-PCR analysis with canine specific ret primers, while ret transcript was detected in mRNA from normal dog testes. (FIG. 1A, B). The ret cDNA from adult dog testis was cloned as described in the Methods. In MDCK clones stably transfected with gfrα1 but not ret, no ret expression was detected either by RT-PCR with the canine ret primers or Western blotting.

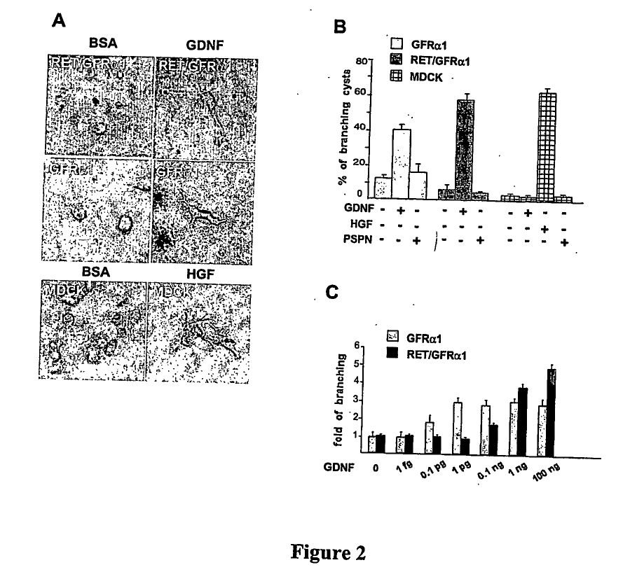

Wild-type MDCK cells form fluid filled cystic structures in three-dimensional (3-D) collagen gels. Following addition of HGF, the cells start forming branching tubules (Montesano et al., 1991). When cultured in the presence of 100 ng/ml GDNF both GFRα1- and RET/GFRα1-expressing MDCK cells formed branching tubules, whereas wild-type MDCK cells did not. (FIG. 2A, B). Mock-transfected cells used as controls also did not from branching tubules in response to GDNF. GDNF also induced branching tubulogenesis in wild-type MDCK transduced with an adenovirus expressing GFRα1 (data not shown). The ligand for GFRα4 (Lindahl et al., 2001), persephin (PSPN), which is not known to interact with GFRα1, did not evoke branching of GFRα1- or RET/GFRα1-expressing MDCK cells.

Both GFRα1-expressing MDCK cells and MDCK cells co-expressing RET and GFRα1 exhibited a dose dependent response to GDNF above a certain threshold concentration (FIG. 2C). The branching response of the cells expressing GFRα1 but not RET was highly sensitive to GDNF, as 0.1 pg/ml of GDNF evoked tubulogenesis (FIG. 2C). In contrast, cells co-expressing RET and GFRα1 started to branch only at 0.1 ng/ml of GDNF (FIG. 2C). GDNF preparations synthesized by different methods and expressed in two different cell types by two different manufactures, were tested (see “Methods”). Both GDNF products differentially evoked branching of GFRα1-expressing MDCK cells at low concentrations (0.1 pg/ml), while the MDCK cells co-expressing RET and GFRα1 started to branch only much higher concentrations of GDNF.

### Example 2

GFRα1-expressing Cells Do Not Respond Chemotactically to GDNF

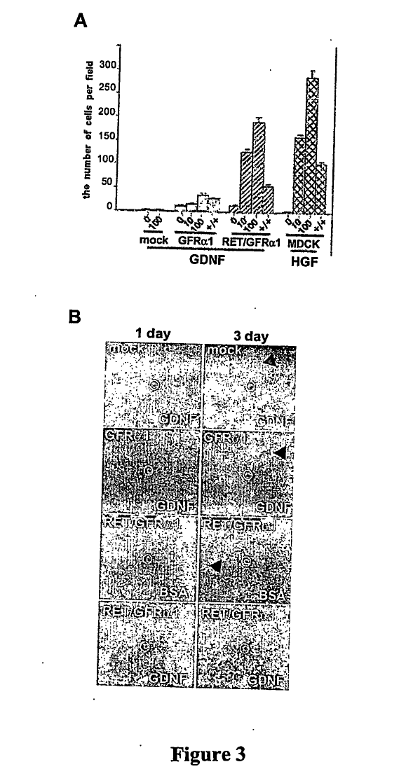

Guided migration of cells towards a chemoattractant is referred to as chemotaxis, whereas enhanced cellular motility is called chemokinesis. Chemotaxis can be tested in the Boyden dual chamber assay by adding the test substance to the lower chamber only, and chemokinesis by adding test substance to both upper and lower chambers. HGF induces chemotaxis of the wild-type MDCK cells (Stoker et al., 1987). Similarly, the chemotactic migration of RET/GFRα1co-expressing MDCK cells is stimulated by GDNF in the presence of soluble GFRα1 (Tang et al., 1998). Accordingly, GDNF induced chemotactic migration of MDCK cells transfected with both RET and the GPI-anchored, membrane bound form of GFRα1. In contrast, the GFRα1-expressing, RET-deficient MDCK cells did not demonstrate a chemotactic response to GDNF under the same conditions (FIG. 3A).

In separate experiments, GDNF or HGF were added to both chambers to test their possible chemokinetic effects on the cell lines. The migration of RET/GFRα1-expressing MDCK cells was reduced threefold as compared to the maximal chemotactic response. Similar reduction was observed with HGF in wild-type MDCK cells. In contrast, the migration of GFRα1-expressing cells was only slightly reduced when GDNF was applied to both chambers (FIG. 3A). Thus, GDNF was chemotactic in RET/GFRα1-expressing cells, but weakly chemokinetic but not chemotactic in GFRα1-expressing, RET-deficient cells.

In another chemotaxis assay, RET/GFRα1-, GFRα1- and mock-transfected MDCK cells were seeded on culture dishes coated with collagen I. Agarose beads were soaked in GDNF (10 ng/μl) or 1% BSA. A bead was placed on top of the soft collagen, and cells were monitored for 3 days. RET/GFRα1-expressing cells actively migrated towards the source of GDNF, but not to BSA (FIG. 3B). Neither GFRα1-expressing, RET deficient cells nor mock-transfected cells, however, were attracted by the GDNF- or BSA-releasing beads (FIG. 3B).

### Example 3

GDNF Activates MET in Both RET-dependent and RET-independent Signaling

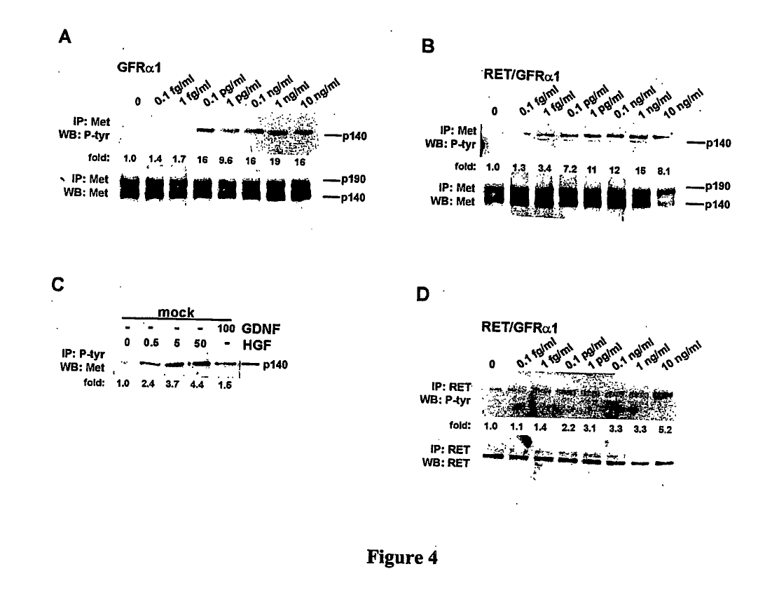

MET is the only receptor known to promote tubule formation in these cells (Santos et al., 1993). Since both GDNF and HGF promoted branching of MDCK cells, experiments were conducted to determine if GDNF can induce MET phosphorylation. GDNF indeed evoked MET phosphorylation in GFRα1- and Ret/GFRα1-expressing MDCK cells, but not in wild type MDCK cells. Dose dependent activation of MET was detected in response to addition of GDNF. Saturation was reached at 0.1 pg/ml (FIG. 4A, B). The same concentration of GDNF also induced rapid MET phosphorylation in human neuroblastoma SHEP cells (data not shown), which express GFRα1 but not RET (Poteryaev et al., 1999). GDNF activated MET in GFRα1-expressing and RET/GFRα1-expressing MDCK cells within 15 minutes (FIG. 4A, B). The activation lasted at least 2 hours (data not shown). In mock-transfected MDCK cells only HGF phosphorylated MET (FIG. 4C).

In RET/GFRα1-expressing MDCK cells, while RET was phosphorylated observed at 0.1 pg/ml of GDNF, saturation was reached at 10 ng/ml (FIG. 4D). RET is phosphorylated in RET-expressing MDCK cells only several hours after GDNF application (Tang et al., 1998). Similarly, no RET activation was observed before 2 hours in these experiments (data not shown). Thus, RET activation by GDNF required higher concentrations and took longer than GDNF activation of MET.

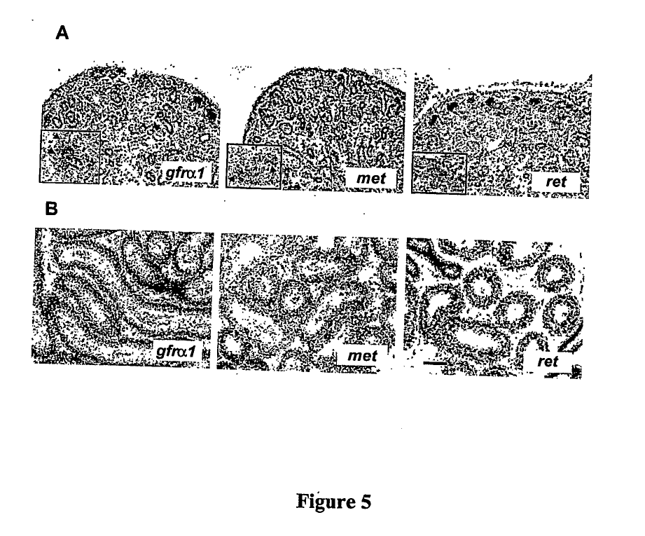

To investigate whether GFRα1 could possibly cooperate with MET in vivo, the tissue distributions of gfrα1, met and ret were compared by in situ hybridization. In E15 kidneys, gfrα1 and met expression overlapped in the tips of the ureteric bud, cap condensates, and pretubular mesenchyme but ret was only found in the ureteric bud tips (FIG. 5A). In the epididymis of adult mice, gfrα1 and met mRNAs are partly co-expressed with ret in the initial segment of epididymal ducts (FIG. 5B), while only gfrα1 and met were expressed in the central body (the corpus) (data not shown).

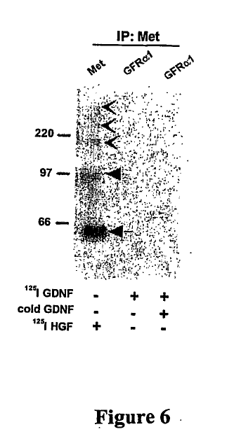

In a series of cross-linking experiments, activation of MET was tested to determine whether the activation by GDNF occurred directly or indirectly. Binding of ¹²⁵I-GDNF to GFRα1-expressing MDCK, SHEP or COS7 cells, and NIH 3T3 cells transiently transfected with GFRα1 was followed by chemical cross-linking and immunoprecipitation with anti-MET antibodies. No high molecular weight complexes were revealed (FIG. 6). Different cross-linkers, such as EDC with sulfo-NHS, BS³, DSS and DSP were tested, each providing the same, negative, results (FIG. 6 and data not shown). Likewise, no direct association of the GDNF receptor complex and MET was detected in RET/ GFRα1-expressing MDCK cells (data not shown).

Cross-linking of ¹²⁵I-HGF in COS7 cells followed by immunoprecipitation with anti-MET antibodies was used as a positive control. It resulted in approximately 200 kDa, 250 kDa and 340 kDa complexes under reducing conditions (FIG. 6). They represent different combinations of the MET β-subunit (140 kDa), Met-αβ heterodimer (190 kDa) and HGF complexes (60 kDa-90 kDa).

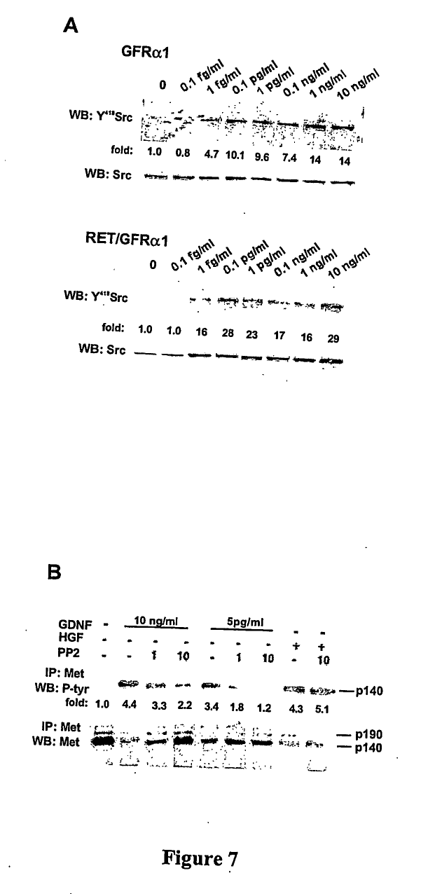

Possible candidates for messengers between GDNF-GFRα1 complex and MET are Src family kinases. Cellular c-Src kinase associates with MET after receptor activation in mink lung epithelial cell line Mv1Lu (Rahimi et al., 1998), and Src-type kinases are activated by GDNF in the GFRα1-dependent, RET-independent signalling (Poteryaev et al., 1999; Trupp et al., 1999). GDNF at 0.1 pg/ml saturated Src phosphorylation in GFRα1-expressing and RET/GFRα1-expressing MDCK cells (FIG. 7A). To detect activated Src, antibodies that specifically recognize phosphorylated residue Tyr418 of activated Src were used (Abram and Courtneidge, 2000).

At a concentration of 1 μM, the Src-type kinase inhibitor, PP2, strongly reduced the GDNF-dependent phosphorylation of MET in SHEP cells (FIG. 7B). In contrast, PP2 did not affect HGF-induced MET phosphorylation (FIG. 7B). Accordingly, PP2 inhibited GDNF-induced MET activation in both GFRα1- and RET/GFRα1-expressing MDCK cells, but not the HGF-induced MET phosphorylation in these cells (data not shown).

### Example 4

Exogenous GDNF Partially Restores the Renal Phenotype of Ret-deficient Mice

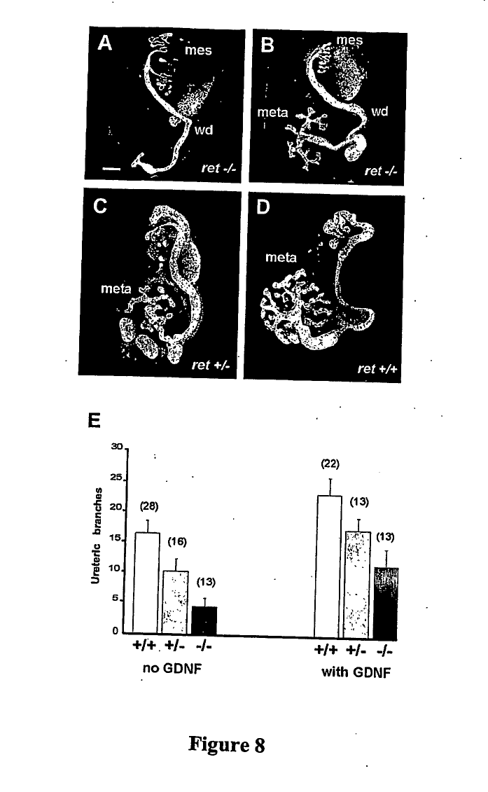

To analyze the possible role of RET-independent, GFRα1-mediated signaling in ureteric budding and branching during nephrogenesis, the ability of exogenous GDNF to induce ureteric budding or sustain its branching in ret-deficient mice was tested. E11 ret −/− urogenital blocks including kidney rudiments were cultured for 4 days with or without 50 ng/ml of GDNF (FIG. 8). As expected, the ureteric buds of ret −/− mice did not branch or branched rudimentarily in the control media (see Schuchardt et al., 1996). When the culture medium was supplemented with GDNF, the number of ureteric bud tips in the hypodysplastic kidneys of ret-deficient mice was increased but not to the level seen in wild type kidneys (FIG. 8B, D). Exogenous GDNF increased the number of ureteric bud tips in ret −/−, ret +/− and wild-type kidney explants (FIG. 8E). However, with or without exogenous GDNF, the number of ret −/− urogenital explants completely lacking a ureteric bud remained the same (Table 1). Thus, RET-independent signaling by GDNF has an apparent role in the ureteric branching, but may be less significant in the primary bud formation.

### DISCUSSION OF EXAMPLES

Novel RET-independent GDNF receptor-effected morphological responses to GDNF are demonstrated. Such morphological responses can conveniently be used as the basis for methods of identifying modulators of RET-independent intracellular signaling as disclosed herein.

The RET-independent GDNF receptor-effected morphological responses can partially restore ureteric branching of ret-deficient hypodysplastic kidneys, when GDNF ligands are applied to the culture medium. GDNF induces branching, but not chemotactic migration of MDCK cells expressing GFRα1 but not RET. Because MET is the only receptor to promote branching of wild type MDCK cells, whether GDNF activates MET in non-RET signaling was explored. Indeed, in MDCK and several other cell types, GDNF binding to GFRα1activates MET indirectly via Src-family kinases. Src activation is essential for both MET activation and branching morphogenesis. These data, together with the overlapping expression patterns of met and gfrα1 in embryonic organs, underline the role of GFRα1 in branching morphogenesis and provide biochemical and biological evidence for a novel signaling mechanism of GDNF.

The development of the mammalian permanent kidney or metanephros requires reciprocal inductive interactions between the metanephric mesenchyme and the ureteric bud (Kuure et al., 2000). GDNF is an essential mesenchymal signal for ureteric budding and branching (Sariola and Saarma, 1999). GDNF has also been assumed to signal during kidney morphogenesis via the GFRα1 and RET complex, because GDNF-soaked beads fail to induce ectopic buds from Wolffian ducts of ret-deficient mice (Sainio et al., 1997) and wild type metanephric mesenchymes co-cultured with ret-deficient ureteric buds do not restore branching (Schuchardt et al. 1996). It is notable that the renal phenotype of mice lacking RET is variable ranging from total aplasia to hypodysplasia. Metanephric development is initiated in 61% of ret-deficient embryos (Schuchardt et al., 1996). Partially restored branching of ret-deficient hypodysplastic kidney rudiments by exogenous supplementation of GDNF has now been accomplished, but thus far, attempts using GDNF to decrease the number of kidney explants with complete renal aplasia have failed. The data suggest that RET-independent signaling via GFRα1 effects sustained ureteric branching rather than bud initiation from the Wolffian duct. The Boyden chamber results with MDCK cells further suggest that RET is critical for chemotactic response to GDNF. This indicates the proper orientation of the tips of the ureteric buds within the nephrogenic mesechyme is critically controlled by RET activity.

GFRα1, a GPI-linked receptor, does not have an intracellular domain. In the absence of RET, GFRα1 may thus employ other transmembrane molecule(s) for signal transduction. The similarity between the GDNF- and HGF-induced branching responses of MDCK cells prompted the study of the role of GDNF as a direct or indirect activator of MET. Indeed, GDNF evoked MET phosphorylation in GFRα1-expressing and RET/GFRα1-expressing cells, but not in wild-type MDCK cells. MET activation by GDNF is not restricted to a particular cell line or cell type, since it takes place in SHEP cells endogenously expressing GFRα1, as well as in GFRα1-transfected COS7 cells and NIH 3T3 fibroblasts. However, the cross-linking experiments utilizing several different chemical cross-linkers and several cell lines failed to detect any GDNF complexes with MET, making a direct interaction between GFRα1 and MET highly improbable.

Src-type kinases may mediate GDNF signalling from GFRα1 to MET, since (i) Src family kinases are associated with the lipid rafts like GFRα1 (Harder et al., 1998); (ii) Src-type kinase is activated in RET-independent signalling by GDNF (Poteryaev et al., 1999; Trupp et al., 1999); (iii) c-Src kinase is associated with MET after receptor activation (Rahimi et al., 1998); (iv) Integrin-mediated activation of RON receptor, homologous to MET, requires c-Src (Danilkovitch-Miagkova et al., 2000). Indeed, inhibition of Src-type kinases by PP2 prevents phosphorylation of MET by GDNF, but not by HGF. Thus, Src kinases are upstream to MET in the GDNF signalling pathway but downstream to MET in HGF signalling.

The kinetics of MET and RET activation by GDNF are different and such differences may be exploited in implementing the methods of the present invention. In the GFRα1-expressing and RET/GFRα1-expressing MDCK cells, Src-type kinases and MET were activated in 15 minutes (and their activity continued at least for 2 hours), while RET was activated only after 2 hours. Thus, at least early MET activation may depend only on GFRα1.

Low concentrations of GDNF result in phosphorylation of MET in the GFRα1-expressing and RET/GFRα1-expressing MDCK cells. Intriguingly, MET is saturated by GDNF at 4 fM (0.1 pg/ml), whereas HGF saturates MET at 0.5 nM (Villa-Moruzzi et al., 1993). The phenomenon of femtomolar concentrations causing activation of a signalling pathway and biological response is not unique: femtomolar levels of GABA neurotransmitter stimulate migration of a subpopulation of cortical neurons (Behar et al., 1996; Hayashi et al., 2002). Similarly, the delta opioid peptide [D-Ala²,D-Leu⁵]enkephalin promotes PC12 cell survival via the MEK-ERK pathway at femtomolar concentrations (Hayashi et al., 2002).

Additionally, low doses of GDNF induce branching, but not chemotaxis of GFRα1-expressing ret-deficient MDCK cells. When RET is present, GDNF induces both branching and chemotaxis, but only at high concentrations. Thus, different doses of GDNF, as well as the receptor context, define the cellular responses to the ligand. Such differences can again be exploited in the use of the methods of the present invention.

Kidney development is normal in MET-deficient and HGF-deficient mice (Birchmeier and Gherardi, 1998), although in organ culture, HGF neutralizing antibodies disrupt kidney development (Woolf et al., 1995). In contrast, GDNF plays a crucial role in kidney differentiation both in vivo and in vitro. The in vivo contribution of GDNF/MET signaling in kidney morphogenesis can be further elucidated, but at least in organ culture, GDNF partially restores branching morphogenesis in ret-deficient kidney rudiments.

Both met and ret are proto-oncogenes. The met gene is upregulated in several different cancer forms (Giordano et al., 2000), and HGF and MET proteins are frequently overexpressed in breast carcinomas (Tuck et al., 1996; Ghoussoub et al., 1998). Activated RET upregulates met in normal human thyrocytes (Ivan et al., 1997). Mutations in met are known in familial papillary renal cancer and in a few cases of sporadic papillary renal cancer (Schmidt et al., 1997; Zhuang et al., 1998). Oncogenic ret mutations cause multiple endocrine neoplasia type 2A and 2B syndromes, familial medullary thyroid cancer and pheochromocytomas (Pasini et al., 1996; Edery et al., 1997). Src kinases are highly expressed in human breast cancer (50) and Src kinase is activated in SP1 carcinoma cells (Rahimi et al., 1998; Rahimi et al., 1996). The sustained activation of Src stimulates strong expression of HGF in carcinoma cells, which may lead to invasiveness and metastasis (Hung and Elliott, 2001). Different cell lines expressing oncogenic forms of RET possess high Src kinase activity levels (Melillo et al., 1999). Thus, the interplay between MET, RET and Src kinases is apparently crucial in carcinogenesis. Because GDNF induced MET phosphorylation in Neuro-2A neuroblastoma cells, the possible oncogenic role of the GDNF-MET interplay in cancer progression should be further elucidated.

During recent years, evidence for cross-talk between heterologous receptor tyrosine kinases and signaling pathways has emerged. A neuromodulator, adenosine, acting through the A_{2A} receptors activates Trk neurotrophin receptors in the absence of their ligands (Lee and Chao, 2001). Binding of nerve growth factor to TrkA promotes phosphorylation of RET in a GDNF-independent manner (Tsui-Pierchala et al., 2001). MET is also activated by factors other than HGF: the Listeria surface protein, InIB, binds to, and phosphorylates, MET (Shen et al., 2000). Additionally, epidermal growth factor receptor activates MET in transformed cells (Bergstrom et al., 2000; Jo et al., 2000). Src family kinases are mediators between receptor complexes (Danilkovitch-Miagkova et al., 2000; Lee and Chao, 2001). Thus, a horizontal activation mechanism of a receptor tyrosine kinase by heterologous ligand/receptor systems may be more common than assumed.

The horizontal activation of MET by GDNF via Src kinases demonstrates a synergy of two signaling systems, which should be taken into consideration when the biological and pathological effects of MET, GFRα1 and RET are studied.

The foregoing examples are meant to illustrate the invention and are not to be construed to limit the invention in any way. Those skilled in the art will recognize modifications that are within the spirit and scope of the invention.

### REFERENCES

All references cited herein are hereby incorporated by reference in their entireties.

Abram, C. L, and Courtneidge, S. A. (2000). Exp. Cell Res. 254, 1-13.

Airaksinen, M. S., Titievsky, A. and Saarma, M. (1999) GDNF family neurotrophic factor signaling: four masters, one servant? Molecular and Cellular Neuroscience, 13, 313-325.

Arénas, E., Trupp, M., Åkerud, P. and Ibañéz, C. F. (1995) GDNF prevents degeneration and promotes the phenotype of brain noradrenergic neurons in vivo. Neuron, 15, 1465-1473.

Baloh, R. H., Enomoto, H., Johnson, E. M. Jr., and Milbrandt, J. (2000)Curr. Opin. Neurobiol. 10, 103-110.

Baloh, R. H., Tansey, M. G., Lampe, P. A., Fahmer, T. J., Enomoto, H., Simburger, K. S., Leitner, M. L., Araki, T., Johnson, E. M. J. and Milbrandt, J. (1998) Artemin, a novel member of the GDNF ligand family, supports peripheral and central neurons and signals through the GFRα3-RET receptor complex. Neuron, 21, 1291-1302.

Beck, K. D., Valverde, J., Alexi, T., Poulsen, K., Moffat, B., Vandlen, R. A., Rosenthal, A. and Hefti, F. (1995) Mesencephalic dopaminergic neurons protected by GDNF from axotomy-induced degeneration in the adult brain. Nature, 373, 339-341.

Behar, T. N, Li, Y. X., Tran, H. T., Ma, W., Dunlap, V., Scott, C., and Barker, J. L. (1996) J. Neurosci. 16, 1808-1818.

Behar, T. N., Schaffner, A. E., Scott, C. A., O'Connell, C., and Barker, J. L. (1998) J. Neurosci. 18, 6378-6387.

Bergstrom, J. D., Westermark, B., and Heldin, N. E. (2000) Exp. Cell Res. 259, 293-299.

Berridge, M. J. (1998) Neuronal calcium signaling. Neuron, 21, 13-26.

Birchmeier, C., and Gherardi, E. (1998) Trends Cell Biol. 8, 404-410.

Borrello, M. G., Alberti, L., Arighi, E., Bongarzone, I., Battistini, C., Bardelli, A., Pasini, B., Piutti, C., Rizzetti, M. G., Mondellini, P., Radice, M. T. and Pierotti, M. A. (1996) The full oncogenic activity of Ret/ptc2 depends on tyrosine 539, a docking site for phospholipase Cgamma. Molecular & Cellular Biology, 16, 2151-2163.

Bourette, R. P., Myles, G. M., Choi, J. L. and Rohrschneider, L. R. (1997) Sequential activation of phoshatidylinositol 3-kinase and phospholipase C-gamma2 by the M-CSF receptor is necessary for differentiation signaling. EMBO Journal, 16, 5880-5893.

Brown, D. A. and London, E. (1998) Functions of lipid rafts in biological membranes. Annual Review of Cell Developmental Biology, 14, 111-136.

Buj-Bello, A., Buchman, V. L., Horton, A., Rosenthal, A. and Davies, A. M. (1995) GDNF is an age-specific survival factor for sensory and autonomic neurons. Neuron, 15, 821-828.

Buj-Bello, A., Adu, J., Piñon, L. G. P., Horton, A., Thompson, J., Rosenthal, A., Chinchetru, M., Buchman, V. L. and Davies, A. M. (1997) Neurturin responsiveness requires a GPI-anchored receptor and the RET receptor tyrosine kinase. Nature, 387, 721-724.

Cacalano, G., Fariñãs, I., Wang, L. C., Hagler, K., Forgie, A., Moore, M., Armanini, M., Phillips, H., Ryan, A. M., Reichardt, L. F., Hynes, M., Davies, A. and Rosenthal, A. (1998) GFRα1 is an essential receptor component for GDNF in the developing nervous system and kidney. Neuron, 21, 53-62.

Chiariello, M., Visconti, R., Carlomagno, F., Melillo, R. M. Bucci, C., de Franciscis, V., Fox, G. M., Jing, S., Coso, O. A., Gutkind, J. S., Fusco, A. and Santoro, M. (1998) Signaling of the RET receptor tyrosine kinase through the c-Jun NH2-terminal protein kinases (JNKS): evidence for a divergence of the ERKs and JNKs pathways induced by Ret. Oncogene, 16, 2435-2445.

Copp, A., and Cockroft, D. (1991) Postimplantation Mammalian Embryos: A practical approach, pp. 155-171, Oxford University Press, London.

Davletov, B. A., Meunier, F. A., Ashton, A. C., Matsushita, H., Hirst, W. D., Lelianova, V. G., Wilkin, G. P., Dolly, J. O. and Ushkaryov, Y. A. (1998) Vesicle exocytosis stimulated by alpha-latrotoxin is mediated by latrophilin and requires both external and stored Ca²⁺. EMBO Journal 17, 3909-3920.

Danilkovitch-Miagkova, A., Angeloni, D., Skeel, A., Donley, S., Lerman, M., and Leonard, E. J. (2000) J. Biol. Chem. 275, 14783-14786.

Dikic, I., Tokiwa, G., Lev, S., Courtneidge, S. A. and Schlessinger, J. (1996) A role for Pyk2 and Src in linking G-protein-coupled receptors with MAP kinase activation. Nature, 383, 547-550.

Durbec, P., Marcos-Gutierrez, C. V., Kilkenny, C., Grigoriou, M., Wartiowaara, K., Suvanto, P., Smith, D., Ponder, B., Constantini, F., and Saarma, M. et al. (1996) GDNF signaling through the RET receptor tyrosine kinase. Nature, 381, 789-793.

Edery, P., Eng, C., Munnich, A., and Lyonnet, S. (1997) Bioessays 19, 389-395.

Enomoto, H., Araki, T., Jackman, A., Heuckeroth, R. O., Snider, W. D., Johnson, E. M., Jr. and Milbrandt, J. (1998) GFR alpha1-deficient mice have deficits in the enteric nervous system and kidneys. Neuron, 21, 317-324.

Finkbeiner, S., Tavazoie, S. F., Maloratsky, A., Jacobs, K. M., Harris, K. M. and Greenberg, M. E. (1997) CREB: A major mediator of neuronal neurotrophin responses. Neuron, 19, 1031-1047.

Finkbeiner, S. and Greenberg, M. E. (1998) Ca²⁺ channel-regulated neuronal gene expression. Journal of Neurobiology, 37, 171-189.

Friedrichson, T. and Kurzchalia, T. V. (1998) Microdomains of GPI-anchored proteins in living cells revealed by crosslinking. Nature, 394, 802-805.

Fukunaga, K. and Miyamoto, E. (1998) Role of MAP kinase in neurons. Molecular Neurobilogy, 16, 79-95.

Ghosh, A. and Greenberg, M. E. (1995) Calcium signaling in neurons: molecular mechanisms and cellular consequences. Science, 268, 239-247.

Ghoussoub, R. A. D., Dillon, D. A., D'Aquila, T., Rimm, B. E., Fearon, E. R., and Rimm, D. L. (1998) Cancer 82, 1513-1520.

Giordano, S., Maffe, A., Williams, T. A., Artigiani, S., Gual, P., Bardelli, A., Basilico, C., Michieli, P., and Comoglio, P. M. (2000) FASEB J. 14, 399-406.

Golden, J. P., DeMaro, J. A., Osborne, P. A., Milbrandt, J. and Johnson, E. M. Jr. (1999) Expression of neurturin, GDNF, and GDNF family-receptor mRNA in the developing and mature mouse. Experimental Neurology, 58, 504-528.

Green, J. M., Schreiber, A. D. and Brown, E. J. (1997) Role for a glycan phosphoinositol anchor in (Fed. Cir. gamma receptor synergy. Journal of Cell Biology, 139, 1209-1217.

Harder, T., Scheiffele, P., Verkade, P. and Simons, K. (1998) Lipid domain structure of the plasma membrane revealed by patching of membrane components. Journal of Cell Biology, 141, 929-942.

Hayashi, T., Tsao, L. I., and Su, T. P. (2002) Synapse 43, 86-94.

Hellmich, H. L., Kos, L., Cho, E. S., Mahon, K. A., and Zimmer, A. (1996) Mech. Dev. 54, 95-105.

Henderson, C. E., Phillips, H. S., Pollock, R. A., Davies, A. M., Lemeulle, C., Armanini, M., Simmons, L., Moffet, B., Vandlen, R. A., Simpson, L. C., Simmons, L. and et al. (1994) GDNF: a potent survival factor for motoneurons present in peripheral nerve and muscle. Science, 266, 1062-1064.

Hishiki, T., Nimura, Y., Isogai, E., Kondo, K., Ichimiya, S., Nakamura, Y., Ozaki, T., Saliyama, S., Hirose, M., Seki, N., Takahashi, H., Ohnuma, N., Tanabe, M. and Nakagawara, A. (1998) Glial cell line-derived neurotrophic factor/neurturin-induced differentiation and its enhancement by retinoic acid in primary human neuroblastomas expressing c-Ret, GFR alpha-1, and GFR alpha-2. Cancer Research, 58, 2158-2165.

Hung, W., and Elliott, B. (2001) J. Biol. Chem. 276, 12395-12403.

Impey, S., Obrietan, K. and Storm, D. R. (1999) Making new connections: role of ERK/MAP kinase signaling in neuronal plasticity. Neuron, 23, 11-14.

Ivan, M., Bond, J. A., Prat, M., Comoglio, P. M., and Wynford-Thomas, D. (1997) Oncogene 14, 2417-2423.

Jiang, H. and Guroff, G. (1997) Actions of the neurotrophins on calcium uptake. Journal of Neuroscience Research, 50, 355-360.

Jing, S. Q., Wen, D. Z., Yu, Y. B., Holst, P. L., Luo, Y., Fang, M., Tamir, R., Antonio, L, Hu, Z., Cupples, R., Louis, J. C., Hu, S., Altrock, B. W. and Fox, G. M. (1996) GDNF-induced activation of the RET protein tyrosine kinase is mediated by GDNFR-alpha, a novel receptor for GDNF. Cell, 85, 1113-1124.

Jing, S., Yu, Y., Fang, M., Hu, Z., Holst, P. L., Boone, T., Delaney, J., Schultz, H., Zhou, R. and Fox, G. M. (1997) GFRα-2 and GFRα-3 are two new receptors for ligands of the GDNF family. Journal of Biological Chemistry, 272, 33111-33117.

Jo, M., Stolz, D. B., Esplen, J. E., Dorko, K., Michalopoulos, G. K., and Strom, S. C. (2000) J. Biol. Chem. 275, 8806-8811.

Karp, S. L., Ortiz-Arduan, A., Li, S., and Neilson, E. G. (1994) Proc. Natl. Acad. Sci. U.S.A. 91, 5286-5290.

Khare, S., Bolt, M. J., Wali, R. K., Skarosi, S. F., Roy, H. K., Niedziela, S., Scaglione-Sewell, B., Aquino, B., Abraham, C., Sitrin, M.D., Brasitus, T. A. and Bissonnette, M. (1997) 1,25 dihydroxyvitamin D3 stimulates phospholipase C-gamma in rat colonocytes: role of c-Src in PLC-gamma activation. Journal of Clinical Investigation, 99, 1831-1841.

Klein, R. D., Sherman, D., Ho, W. H., Stone, D., Bennett, G. L., Moffat, B., Vandlen, R., Simmons, L., Gu, Q., Hongo, J. A. et al. (1997) A GPI-linked protein that interacts with RET to form a candidate neurturin receptor. Nature, 387, 717-721.

Kokaia, Z., Airaksinen, M. S., Nanobashvili, A., Larsson, E., Kujamäki, E., Lindvall, O. and Saarma, M. (1999) GDNF family ligands and receptors are differentially regulated after brain insults in the rat. European Journal of Neuroscience, 11, 1202-1216.

Kotzbauer, P. T., Lampe, P. A., Heuckeroth, R. O., Golden, J. P., Creedon, D. J., Johnson, E M J and Milbrandt, J. (1996) Neurturin, a relative of glial-cell-line-derived neurotrophic factor. Nature, 384, 467-470.

Kuure, S., R. Vuolteenaho, and S. Vainio. (2000) Kidney morphogenesis: cellular and molecular regulation. Mech. Dev. 92:31-45.

Lang, D. M., Lommel, S., Jung, M., Ankerhold, R., Petrausch, B., Laessing, U., Wiechers, M. F., Plattner, H. and Stuermer, C. A. O. (1998) Identification of reggie-1 and reggie-2 as plasmamembrane-associated proteins which cocluster with activated GPI-anchored cell adhesion molecules in non-caveolar micropatches in neurons. Journal of Neurobiology, 37, 502-523.

Lee, F. S., and Chao, M. V. (2001) Proc. Natl. Acad. Sci. U.S.A. 98, 3555-3560.

Lin, L. F., Doherty, D. H., Lile, J. D., Bektesh, S. and Collins, F. (1993) GDNF: a glial cell line-derived neurotrophic factor for midbrain dopaminergic neurons. Science, 260, 1130-1132.

Lindahl, M., Poteryaev, D., Liying, Y., Arumäe, U., Timmusk, T., Bongarzone, I., Aiello, A., Pierotti, M. A., Airaksinen, M. S., and Saarma, M. (2001). J. Biol. Chem. 276, 9344-9351.

Luttrell, L. M., Hawes, B. E., van Biesen, T., Luttrell, D. K., Lansing, T. J., Lefkowitz, R. J. (1996) Role of c-Src tyrosine kinase in G protein-coupled receptor- and G betagamma subunit-mediated activation of mitogen-activated protein kinases. Journal of Biological Chemistry, 271, 19443-19450.

Luttrell, L. M., Daaka, Y., Della Rocca, G. J. and Lefkowitz, R. J. (1997) G protein-coupled receptors mediate two functionally distinct pathways of tyrosine phosphorylation in rat 1a fibroblasts. Shc phosphorylation and receptor endocytosis correlate with activation of Erk kinases. Journal of Biological Chemistry, 272, 31648-31656.

Luttrell, L. M., Ferguson, S. S. G., Daaka, Y., Miller, W. E., Maudsley, S., Della Rocca, G. J., Lin, F.-T., Kawakatsu, H., Owada, K., Luttrell, D. K., Caron, M. G. and Lefkowitz, R. J. (1999) b-arrestin-dependent formation of b2 adrenergic receptor-Src protein kinase complexes. Science, 283, 655-661.

Melillo, R. M., Barone, M. V., Lupoli, G., Cirafici, A. M., Carlomagno, F., Visconti, R., Matoskova, B., Di Fiore, P. P., Vecchio, G., Fusco, A., and Santoro, M. (1999) Cancer Res. 59, 1120-1126.

Meng, X., Lindahl, M., Hyvönen, M. E., Parvinen, M., de Rooij, D. G., Hess, M. W., Raatikainen-Ahokas, A., Sainio, K., Rauvala, H., Lakso, M., Pichel, J. G., Westphal, H., Saarma, M., and Sariola, H. (2000) Science 287, 1489-1493.

Meyer Z, Heringdorf D., Lass H., Alemany R., Laser K. T., Neumann E., Zhang C., Schmidt M., Rauen U., Jakobs K. H., van Koppen C. J. (1998) Sphingosine kinase-mediated Ca²⁺ signaling by G-protein-coupled receptors. EMBO Journal, 17, 2830-2837.

Milbrandt, J., de Sauvage, F. J., Fahrner, T. J., Baloh, R. H., Leitner, M. L., Tansey, M G, Lampe, P. A., Heuckeroth, R. O., Kotzbauer, P. T., Simburger, K. S., Golden, J. P., Davies, J. A., Vejsada, R., Kato, A. C., Hynes, M., Sherman, D., Nishimura, M., Wang, L C, Vandlen, R., Moffat, B., Klein, R. D., Poulsen, K., Gray, C., Garces, A. and Johnson, E. M. J. (1998) Persephin, a novel neurotrophic factor related to GDNF and neurturin. Neuron, 20, 245-253.

Montesano, R., Matsumoto, K., Nakamura, T., and Orci, L. (1991) Cell 67, 901-908.

Moore, M. W., Klein, R. D., Farinas, I., Sauer, H., Armanini, M., Phillips, H., Reichardt, L. F., Ryan, A. M., Carver-Moore, K. and Rosenthal, A. (1996) Renal and neuronal abnormalities in mice lacking GDNF. Nature, 382, 76-79.

Naldini, L., Vigna, E., Narsimhan, R. P., Gaudino, G., Zarnegar, R., Michalopoulos, G. K., and Comoglio, P. M. (1991) Oncogene 6, 501-504.

Natarajan, D., Grigoriou, M., Marcos-Gutierrez, C. V., Atkins, C. and Pachnis, V. (1999) Multipotential progenitors of the mammalian enteric nervous system capable of colonising aganglionic bowel in organ culture. Development, 126, 157-168.

Oppenheim, R. W., Houenou, L. J., Johnson, J. E., Lin, L. F., Li, L., Lo, A. C., Newsome, A L, Prevette, D. M. and Wang, S. (1995) Developing motor neurons rescued from programmed and axotomy-induced cell death by GDNF. Nature, 373, 344-346.

Ottenhoff-Kalff, A. E., Rijksen, G. A. U., Hennipman, A., Michels, A. A., and Staal, G. E. (1992) Cancer Res. 52, 4773-4778.

Pachnis, V., Mankoo, B., and Costantini, F. (1993) Development 119, 1005-1017.

Paratcha, G., Ledda, F., Baars, L., Coulpier, M., Besset, V., Anders, J., Scott, R., and Ibáñez, C. F. (2001) Neuron 29, 171-184.

Pasini, B., Ceccherini, I., and Romeo, G. (1996) Trends Genet. 12, 138-144.

Pichel, J. G., Shen, L., Sheng, H. Z., Granholm, A. C., Drago, J., Grinberg, A., Lee, E J, Huang, S. P., Saarma, M., Hoffer, B. J., Sariola, H. and Westphal, H. (1996) Defects in enteric innervation and kidney development in mice lacking GDNF. Nature, 382, 73-76.

Poteryaev, D., A. Titievsky, Y. F. Sun, J. Thomas-Crusells, M. Lindahl, M. Billaud, U. Aurumäe, and M. Saarma. (1999) GDNF triggers a novel RET-independent Src kinase family-coupled signaling via a GPI-linked GDNF receptor α1. FEBS Lett. 463:63-66.

Rahimi, N., Hung, W., Tremblay, E., Saulnier, R., and Elliott, B. (1998) J. Biol. Chem. 273, 33714-33721.

Rahimi, N., Tremblay, E., McAdam, L., Park, M., Schwall, R., and Elliott, B. E. (1996) Cell Growth Differ. 7, 263-270.

Rossi, J., Luukko, K., Poteryaev, D., Laurikainen, A., Sun, Y. F., Laakso, T., Eerikäinen, S., Tuominen, R., Lakso, M., Rauvala, H., Arumäe, U., Pasternack, M., Saarma, M. and Airaksinen, M. S. (1999) Retarded growth and deficits in the enteric and parasympathetic nervous system in mice lacking GFRα2, a functional neurturin receptor. Neuron, 22, 243-252.

Saarma, M. (2001) Trends Neurosci. 24, 427-429.

Saarma, M. and Sariola, H. (1999) Other neurotrophic factors: glial cell line-derived neurotrophic factor (GDNF). Microscopy Research Techniques, 45, 292-302.

Sainio, K., P. Suvanto, J. Davies, J. Wartiovaara, K. Wartiovaara, M. Saarma, U. Aurumäe, X. Meng, M. Lindahl, V. Pachnis, and H. Sariola. (1997) Glial-cell-line-derived neurotrophic factor is required for bud initiation from ureteric epithelium. Development 124:4077-4087.

Sambrook, J., Fritsch, E. F., and Maniatis, T. (1989) Molecular Cloning: A Laboratory Manual, 2nd Ed., Cold Spring Harbor Laboratory Press, Cold Spring Harbor, N.Y.

Sanicola, M., Hession, C., Worley, D., Carmillo, P., Ehrenfels, C., Walus, L., Robinson, S., Jaworski, G., Wei, H., Tizard, R., Whitty, A., Pepinsky, R. B. and Cate, R. L. (1997) Glial cell line-derived neurotrophic factor-dependent RET activation can be mediated by two different cell-surface accessory proteins. Proc. Nat. Acad. Sci. USA, 94, 6238-6243.

Sánchez, M. P., Silos-Santiago, I., Frisen, J., He, B., Lira, S. A. and Barbacid, M. (1996) Renal agenesis and the absence of enteric neurons in mice lacking GDNF. Nature, 382, 70-73.

Santos, O. F., L. A. Moura, E. M. Rosen, and S. K. Nigam. (1993) Modulation of HGF-induced tubulogenesis and branching by multiple phosphorylation mechanisms. Dev. Biol. 159:535-548.

Sargiacomo, M., Sudol, M., Tang, Z. and Lizanti, M. P. (1993) Signal transducing molecules and glycosyl-phosphatidylinositol-linked proteins form a caveolin-rich insoluble complex in MDCK cells. Journal of Cell Biology, 122, 789-807.

Sariola, H., and M. Saarma. 1999. GDNF and its receptors in the regulation of the ureteric branching. Int. J. Dev. Biol. 43:413-418.

Schmidt, L., Duh, F. M., Chen, F., Kishida, T., Glenn, G., Choyke, P., Scherer, S. W., Zhuang, Z., Lubensky, I., Dean, M., et al. (1997) Nat. Genet. 16, 68-73.

Schuchardt A, V. D'Agati, V. Pachnis, and F. Costantini. (1996) Renal agenesis and hypodysplasia in ret-k-mutant mice result from defects in ureteric bud development. Development 122:1919-1929.

Schuchardt, A., D'Agati, V., Larsson-Blomberg, L., Costantini, F. and Pachnis, V. (1994) Defects in the kidney and enteric nervous system of mice lacking the tyrosine kinase receptor Ret. Nature, 367, 380-383.

Sharenberg, A. M. and Kinet, J.-P. (1998) PtdIns-3,4,5-P3: a regulatory nexus between tyrosine kinases and sustained calcium signals. Cell, 94, 5-8.

Shen, Y., Naujokas, M., Park, M., and Ireton, K. (2000) Cell 103, 501-510.

Simons, K., and Toomre, D. (2000) Nat. Rev. Mol. Cell. Biol. 1, 31-39.

Simons, K. and Ikonen, E. (1997) Functional rafts in cell membranes. Nature, 387, 569-572.

Stam, J. C., Michiels, F., van der Kammen, R. A., Moolenaar, W. H. and Collard, J. G. (1998) Invasion of T-lymphoma cells: cooperation between Rho family GTPases and lysophospholipid receptor signaling. EMBO Journal, 17, 4066-74.

Stoker, M., Gherardi, E., Perryman, M., and Gray, J. (1987) Nature 327, 239-242.

Suvanto, P., Wartiovaara, K., Lindahl, M., Arumäe, U., Moshnyakov, M., Horelli-Kuitunen, N., Airaksinen, M. S., Palotie, A., Sariola, H. and Saarma, M. (1997) Cloning, mRNA distribution and chromosomal localisation of the gene for glial cell line-derived neurotrophic factor receptor b, a homologue to GDNFR-a. Human Molecular Genetics, 6, 1267-1273.

Suvanto, P., Hiltunen, J. O., Arumäe, U., Moshnyakov, M., Sariola, H., Sainio, K., and Saarma, M. (1996) Eur. J. Neurosci. 8, 816-822.

Takei, K., Shin, R. M., Inoue, T., Kato, K. and Mikoshiba, K. (1998) Regulation of nerve growth mediated by inositol 1,4,5-trisphosphate receptors in growth cones. Science, 282, 1705-1708.

Tang, M.-J., D. Worley, M. Sanicola, and G. Dressler. (1998) The RET-glial cell-derived neurotrophic factor (GDNF) pathway stimulates migration and chemoattraction of epithelial cells. J. Cell Biol. 142:1337-1345.

Taraviras, S., Marcos-Gutierrez, C. V., Durbec, P., Jani, H., Grigoriou, M., Sukumaran, M., Wang, L. C. Hynes, M., Raisman, G. and Pachnis, V. (1999) Signaling by the RET receptor tyrosine kinase and its role in the development of the mammalian enteric nervous system. Development, 126, 2785-2797.

Thomas, S. M. and Brugge, J. S. (1997) Cellular functions regulated by Src family kinases. Annual Review of Cell & Developmental Biology, 13, 513-609.

Thorn, P., Lawrie, A. M., Smith, P. M., Gallacher, D. V. and Petersen, O. H. (1993) Local and global cytosolic Ca²⁺ oscillations in exocrine cells evoked by agonists and inositol trisphosphate. Cell, 74, 661-668.

Tomac, A., Lindqvist, E., Lin, L. F., Ogren, S. O., Young, D., Hoffer, B. J. and Olson, L. (1995) Protection and repair of the nigrostriatal dopaminergic system by GDNF in vivo Nature, 373, 335-339.

Treanor, J. J., Goodman, L., de Sauvage, F., Stone, D. M., Poulsen, K. T., Beck, C. D., Gray, C., Armanini, M. P., Pollock, R. A., Hefti, F., Phillips, H. S., Goddard, A., Moore, M. W., Buj-Bello, A., Davies, A. M., Asai, N., Takahashi, M., Vandlen, R., Henderson, C. E. and Rosenthal, A. (1996) Characterization of a multicomponent receptor for GDNF. Nature, 382, 80-83.

Trupp, M., Ryden, M., Jörnvall, H., Funakoshi, H., Timmusk, T., Arenas, E. and Ibañéz, C. F. (1995) Peripheral expression and biological activities of GDNF, a new neurotrophic factor for avian and mammalian peripheral neurons. Journal of Cell Biology, 130, 137-148.

Trupp, M., Arenas, E., Fainzilber, M., Nilsson, A. S., Sieber, B. A., Grigoriou, M., Kilkenny, C., Salazar-Grueso, E., Pachnis, V., Arumäe, U., Sariola, H., Saarma, M. and Ibañéz, C. F. (1996) Functional receptor for GDNF encoded by the c-ret proto-oncogene. Nature, 381, 785-788.

Trupp, M., Belluardo, N., Funakoshi, H., Ibañéz, C. F. (1997) Complementary and overlapping expression of glial cell line- derived neurotrophic factor (GDNF), c-RET proto-oncogene, and GDNF receptor-alpha indicates multiple mechanisms of trophic actions in the adult rat CNS. Journal of Neuroscience, 17, 3554-3567.

Trupp, M., Scott, R., Whittemore, S.R. and Ibañéz, C. F. (1999) RET-dependent and -independent mechanisms of glial cell line-derived neurotrophic factor signaling in neuronal cells. Journal of Biological Chemistry, 274, 20885-20894.

Tsui-Pierchala, B. A., Milbrandt, J., and Johnson, E. M. Jr. (2002) Neuron 33, 261-273.

Tuck, A. B., Park, M., Stems, E. E., Boag, A., and Elliott, B. E. (1996) Am. J. Pathol. 148, 225-232.

Usachev, Y. M. and Thayer, S. A. (1999) Ca²⁺ influx in resting rat sensory neurones that regulates and is regulated by ryanodine-sensitive Ca²⁺ stores. Journal of Physiology, 519, 115-130.

Varma, R. and Mayor, S. (1998) GPI-anchored proteins are organized in submicron domains at the cell surface. Nature, 394, 798-801.

Vega, Q. C., Worby, C. A., Lechner, M. S., Dixon, J. E., and Dressler, G. R. (1996) Proc. Natl. Acad. Sci. U.S.A. 93, 10657-10661.

Villa-Morunzzi, E., Lapi, S., Prat, M., Gaudino, G., and Comoglio, P. M. (1993) J. Biol. Chem. 268, 18176-18180.

Viola, A., Schroeder, S., Sakakibara, Y. and Lanzavecchia, A. (1999) T lymphocyte costimulation mediated by reorganization of membrane microdomains. Science, 283, 680-682.

Woolf, A. S., Kolatsi-Joannou, M., Hardman, P., Andermarcher, E., Moorby, C., Fine, L. G., Jat, P. S., Noble, M. D., and Gherardi, E. (1995) J. Cell Biol. 128, 171-184.

Yan, Q., Matheson, C. and Lopez, O. T. (1995) In vivo neurotrophic effects of GDNF on neonatal and adult facial motor neurons. Nature, 373, 341-344.

Ylikoski, J., Pirvola, U., Virkkala, J., Suvanto, P., Liang, X.-Q., Magal, E., Altschuler, R., Miller, J. M. and Saarma, M. (1998) Guinea pig auditory neurons are protected by glial cell line-derived growth factor from degeneration after noise trauma. Hearing Research, 124, 17-26.

Yu, T., Scully, S., Yu, Y., Fox, G. M., Jing, S. and Zhou, R. (1998) Expression of GDNF family receptor components during development: implications in the mechanisms of interaction. Journal of Neuroscience, 18, 4684-4696.

Zhuang, Z., Park, W. S., Pack, S., Schmidt, L., Vortmeyer, A. O., Pak, E., Pham, T., Weil, R. J., Candidus, S., Lubensky, I. A., Linehan, W. M., Zbar, B., and Weirich, G. (1998) Nature Genet, 20, 66-69.

What is claimed is:

1. A method of identifying a compound that modulates RET-independent intracellular signaling effected by a nonRET GDNF receptor in a cell, comprising the steps of:
(a) incubating a cell expressing a nonRET GDNF receptor with a test compound;
(b) measuring an indicator of MET activation; and, (c) comparing the measured indicator to that of a control incubated without the test compound, thereby identifying whether the compound modulates RET-independent intracellular signaling.

2. The method of claim 1 wherein the indicator of MET activation is a RET-independent GDNF receptor-effected morphological response.

3. The method of claim 1 wherein the RET-independent GDNF receptor-effected morphological response is tubulogenesis, branching tubulogenesis, chemotaxis, or chemokinesis.

4. The method of claim 1 wherein the nonRET GDNF receptor is a GFRα receptor.

5. The method of claim 4 wherein the GFRα receptor is a GFRα1 receptor.

6. The method of claim 1 wherein the cell has a phenotype consistent with having no functional RET protein.

7. The method of claim 6 wherein the cell does not contain a RET gene, or contains a defective RET gene.

8. The method of claim 6 wherein the cell expresses a nonfunctional RET protein.

9. The method of claim 1 wherein the cell is a kidney cell, nervous system cell, fibroblast, testicular cell, or epithelial cell.

10. The method of claim 9 wherein the cell has one or more oncogenic mutations.

11. The method of claim 9 wherein the cells is a SHEP neuroblastoma cell, COS7 cell, NIH 3T3 cell, or MDCK dog kidney cell.

12. The method of claim 1 wherein the step of incubating is performed in the presence of a GDNF ligand.

13. The method of claim 12 wherein the GDNF ligand is present in a concentration sufficient to effect RET-independent intracellular signaling.

14. The method of claim 12 wherein the GDNF ligand is present in a concentration sufficient to effect RET-independent intracellular signaling, but below a concentration required for a RET-dependent signaling.

15. The method of claim 12 wherein the GDNF ligand is present at a concentration of less than about 100 pg/ml.

16. The method of claim 12 wherein the GDNF ligand is present at a concentration of less than about 10 pg/ml.

17. The method of claim 12 wherein the GDNF ligand is present at a concentration of less than about 1 pg/ml.

18. The method of claim 12 wherein the GDNF ligand is present at a concentration of less than about 100 fg/ml.

19. The method of claim 12 wherein the GDNF ligand is present at a concentration of less than about 50 fg/ml.

20. The method of claim 2 wherein the step of incubating is performed under conditions wherein the cell does not express a functional RET protein.

21. The method of claim 2 wherein the step of incubating is performed under conditions wherein the function of a RET protein is inhibited.

22. The method of claim 2 wherein the measuring step comprises measuring the morphological response after an incubation time which is less than the incubation time required for RET-dependent GDNF receptor-effected intracellular signaling.

23. The method of claim 22 wherein the morphological response is measured less than about one hour after the incubating is initiated.

24. The method of claim 22 wherein the indicator of morphological response is measured less than about 30 minutes after the incubating is initiated.

25. The method of claim 22 wherein the indicator of morphological response is measured less than about 15 minutes after the incubating is initiated.

26. The method of claim 22 wherein the indicator of morphological response is measured less than about 5 minutes after the incubating is initiated.

27. The method of claim 2 further comprising the step of: (d) comparing the measured morphological response to that of a control assay incubated under conditions which provide a RET-dependent GDNF receptor-effected morphological response, thereby further identifying a compound that modulates RET-independent intracellular signaling, RET-dependent intracellular signaling, or a combination of RET-independent and RET-dependent signaling.

28. A method of identifying a compound that modulates RET-independent intracellular signaling effected by a nonRET GDNF receptor in a cell, comprising the steps of:
(a) incubating a cell expressing a nonRET GDNF receptor with a test compound;
(b) measuring a RET-independent GDNF receptor-effected morphological response; and, (c) comparing the measured response to that of a control assay incubated without the test compound, thereby identifying whether the compound modulates RET-independent intracellular signaling.

29. The method of claim 28 wherein the RET-independent GDNF receptor-effected morphological response is tubulogenesis, branching tubulogenesis, chemotaxis, or chemokinesis.

30. The method of claim 28 wherein the nonRET GDNF receptor is a GFRα receptor.

31. The method of claim 30 wherein the GFRα receptor is a GFRα1 receptor.

32. The method of claim 28 wherein the cell has a phenotype consistent with having no functional RET protein.

33. The method of claim 28 wherein the cell is a kidney cell, nervous system cell, fibroblast, testicular cell, or epithelial cell.

34. The method of claim 33 wherein the cell has one or more oncogenic mutations.

35. The method of claim 33 wherein the cell is a SHEP neuroblastoma cell, COS7 cell, NIH 3T3 cell, or MDCK dog kidney cell.

36 The method of claim 28 wherein the step of incubating is performed in the presence of a GDNF ligand.

37. The method of claim 36 wherein the GDNF ligand is present in a concentration sufficient to effect RET-independent intracellular signaling.

38. The method of claim 36 wherein the GDNF ligand is present in a concentration sufficient to effect RET-independent intracellular signaling but below a concentration required for a RET-dependent signaling.

39. The method of claim 36 wherein the GDNF ligand is present at a concentration of less than about 50 fg/ml.

40. The method of claim 28 wherein the step of incubating comprises conditions wherein the cell does not express a functional RET protein.

41. The method of claim 28 wherein the step of incubating comprises conditions wherein the function of a RET protein is inhibited.

42. The method of claim 28 wherein the measuring step comprises measuring the morphological response after an incubation time which is less than the incubation time required for RET-dependent GDNF receptor-effected intracellular signaling.

43. The method of claim 42 wherein the morphological response is measured less than about 15 after the incubating is initiated.

44. A method of differentiating one or more intracellular signals as effected by one or more receptors for a shared ligand comprising the steps of:
(a) interacting one or more receptors with a ligand at a first concentration for a first duration;
(b) measuring a change in response to the ligand in a first intracellular signal after the first duration;
(c) optionally, continuing to periodically measure changes in the first intracellular signal;
(d) further interacting the one or more receptors with the ligand at a second concentration for a second duration; wherein the second concentration is measurably higher than the first concentration and the second duration is at least as long as the first duration;
(e) measuring a change in response to the ligand in a second intracellular signal after the second duration; and, (f) correlating the first intracellular signal with the first concentration and first duration and the second intracellular signal with the second concentration with the second duration, thereby differentiating the intracellular signals as effected by the one or more receptors.

45. The method of claim 44 wherein the one or more receptors comprise at least one receptor kinase.

46. The method of claim 45 wherein the one or more receptors comprise a GPI-anchored receptor.

47. The method of claim 46 wherein the receptor is a GDNF receptor.

48. The method of claim 47 wherein the receptor is a GFRα.

49. The method of claim 48 wherein the receptor is GFRα1.

50. The method of claim 49 wherein the one or more receptors further comprise a RET.

51. The method of claim 50 wherein the ligand is a GDNF ligand.

52. The method of claim 44 wherein the first concentration of the ligand is less than about 100 pg/ml.

53. The method of claim 44 wherein the first concentration of the ligand is less than about 10 pg/ml.

54. The method of claim 44 wherein the first concentration of the ligand is less than about 1 pg/ml.

55. The method of claim 44 wherein the first concentration of the ligand is less than about 100 fg/ml.

56. The method of claim 44 wherein the first concentration of the ligand is less than about 50 fg/ml.

57. The method of claim 44 wherein the second concentration of the ligand is at least about ten times greater than the first concentration.

58. The method of claim 44 wherein the second concentration of the ligand is at least about twenty times greater than the first concentration.

59. The method of claim 44 wherein the second concentration of the ligand is at least about fifty times greater than the first concentration.

60. The method of claim 44 wherein the second concentration of the ligand is at least about one hundred or more times greater than the first concentration.

61. The method of claim 44 wherein the first and second intracellular signals are the same intracellular signal.

62. The method of claim 44 wherein the intracellular signal comprises MET activation, Src kinase activation.

63. The method of claim 44 wherein the first intracellular signal comprises an Src kinase activation and the Src kinase activation results in an activation of MET.

64. The method of claim 63 wherein the activation of MET can be distinguished from a RET-dependent activation based on a receptor context, concentration of ligand, or time course of activation.

65. The method of claim 64 wherein the activation of MET is inhibited by inhibitors of Src kinases and can be distinguished from MET activation by HGF ligands.

66. The method of claim 64 wherein neither the ligand nor the receptor are associated with the MET.

67. A method of determining, in a cell, interrelationships between MET, RET and Src kinases mediated by multifunctional GDNF ligands comprising the steps of:
(a) incubating a cell with a GDNF ligand at a concentration to be tested under conditions to be tested;
(b) measuring the activation of Src kinases and MET;
(c) correlating the activation of the Src kinases and MET with the concentration of GDNF ligand tested, and with the conditions to be tested, wherein the conditions to be tested include but are not limited to presence or absence of RET-dependent activation mechanisms, presence or absence of HGF ligand-mediated activation of MET, presence or absence of inhibitors of Src kinase; time course of the activation, cell genotype, and cell phenotype; thereby determining the interrelationships between MET, RET and Src kinases as mediated by GDNF ligands.

68. The method of claim 67 wherein the GDNF ligand does not directly associate with, or bind to, the MET.

69. The method of claim 67 wherein the cell is from a patient.

70. The method of claim 69 wherein the patient has a cancer.

71. The method of claim 70 wherein the cancer is a breast cancer, renal cancer, multiple endocrine neoplasia type 2A or 2B syndrome, medullary thyroid cancer, pheochromocytoma, or SP1 carcinoma.

72. A method of identifying putative modulators of interrelationships between MET, RET and Src kinases mediated by GDNF ligands comprising the steps of:
(a) practicing the method of claim 67 in the presence and absence of putative modulators of the interrelationships between MET, RET and Src kinase;
(b) correlating the results in the presence of the putative modulator with those from the results in the absence of the modulator in control assays where all other factors are constant; thereby identifying modulators of the interrelationship.

73. The method of claim 72 wherein the cell is from a patient with a cancer selected from the group consisting of breast cancer, renal cancer, multiple endocrine neoplasia type 2A or 2B syndrome, medullary thyroid cancer, pheochromocytoma, or SP1 carcinoma.


SEQUENCE LISTING (9 sequences)

SEQ ID NO: 1 - DNA, 29 bp, Artificial Sequence
  Description of Artificial Sequence Synthetic primer
aattgctagcgtgagcaagggcgaggagc

SEQ ID NO: 2 - DNA, 29 bp, Artificial Sequence
  Description of Artificial Sequence Synthetic primer
aattgctagcttacttgtacagctcgtcc

SEQ ID NO: 3 - DNA, 32 bp, Artificial Sequence
  Description of Artificial Sequence Synthetic primer
aattgctagcgaccgtctggactgtgtgaaag

SEQ ID NO: 4 - DNA, 30 bp, Artificial Sequence
  Description of Artificial Sequence Synthetic primer
tatagctagctccaccactcacctcggcgg

SEQ ID NO: 5 - DNA, 21 bp, Artificial Sequence
  Description of Artificial Sequence Synthetic primer
agacgtggtacctgcatcagg

SEQ ID NO: 6 - DNA, 20 bp, Artificial Sequence
  Description of Artificial Sequence Synthetic primer
cgttgaagtggagcaagagg

SEQ ID NO: 7 - DNA, 281 bp, Canis familiaris
agacgtgggacctgcatcaggggagctcctgaggcgatacacaagtacactactccctggggatgcctgggccctccagacatttcgtgtggaacactcacccaacgagaccttggtccaggccaacggcagctttgcgcgggcaactgtgcatgactacaggctggttctcaaccggagcctccccatctcggagagccgctccctgcagctggccgttctggtcaatgactcggacttccagggtcccggggagggcatcctcttgctccacttcaacg

SEQ ID NO: 8 - DNA, 21 bp, Artificial Sequence
  Description of Artificial Sequence Synthetic primer
ctgaggcgatacacaagtaca

SEQ ID NO: 9 - DNA, 20 bp, Artificial Sequence
  Description of Artificial Sequence Synthetic primer
tcaatgactcggacttccag